(19) United States
(12) Patent Application Publication
(10) Pub. No.: US 2021/0338648 A1
(43) Pub. Date: Nov. 4, 2021

(54) METHODS AND COMPOSITIONS FOR REDUCING SERUM URIC ACID

(71) Applicant: AstraZeneca AB, Södertälje (SE)

(72) Inventors: Johan HOEGSTEDT, Wilmington, DE (US); James MACKAY, Wilmington, DE (US); Eva JOHNSSON, Södertälje (SE)

(21) Appl. No.: 17/280,929

(22) PCT Filed: Oct. 1, 2018
(86) PCT No.: PCT/IB2018/057612

§ 371 Date: Mar. 29, 2021

(51) Int. Cl. A61K 31/4418; A61K 31/351; A61K 31/426; A61K 31/519; A61P 13/02

(52) U.S. Cl. CPC A61K 31/4418; A61K 31/351; A61P 13/02; A61K 31/519; A61K 31/426

(57) ABSTRACT

The present disclosure provides compositions comprising verinurad, a xanthine oxidase inhibitor, and dapagliflozin useful in the reduction of serum uric acid levels, formulations containing them, and methods using them. In some embodiments, the methods and compositions described herein are used in the treatment or prevention of conditions associated with hyperuricemia, such as chronic kidney disease, heart failure, and gout.

### BACKGROUND

Uric acid is a product of the metabolic breakdown of purine nucleotides. Most uric acid dissolves in blood and passes to the kidneys, where it is excreted by glomerular filtration and tubular secretion. A substantial fraction of uric acid is reabsorbed by the renal tubules. A uric acid concentration in blood plasma above the normal range is known as hyperuricemia. Hyperuricemia has been associated with chronic kidney disease and renal dysfunction, and identified as an independent risk factor for renal function decline. See Levy, G. D., et al. J. Rheum. 2014; 41(5): 955; X. Su et al. PLoS ONE 2017; 12(11): e0187550. Hyperuricemia has also been associated with cardiovascular disease and heart failure. See M. Li, et al. Sci. Rep. 2016; 6: 19520. Thus, a need exists for therapeutic methods and compositions for reducing serum uric acid levels that may be used in therapeutic and prophylactic methods, e.g. to treat or prevent conditions associated with hyperuricemia such as chronic kidney disease and heart failure.

### SUMMARY OF THE INVENTION

In some embodiments, the instant disclosure provides methods of reducing serum uric acid levels in a subject in need thereof, comprising administering to the subject: a URAT1 inhibitor; a xanthine oxidase inhibitor; and an SGLT2 inhibitor. In some embodiments, disclosed herein is a method of reducing serum uric acid levels in a subject in need thereof, comprising administering to the subject: verinurad or a pharmaceutically acceptable salt thereof; a xanthine oxidase inhibitor; and dapagliflozin. In some embodiments, disclosed herein is a method of treating or preventing a condition associated with hyperuricemia in a subject in need thereof, comprising administering to the subject: verinurad or a pharmaceutically acceptable salt thereof; a xanthine oxidase inhibitor; and dapagliflozin. In some embodiments, the condition is gout, a recurrent gout attack, gouty arthritis, hypertension, a cardiovascular disease, coronary heart disease, heart failure, Lesch-Nyhan syndrome, Kelley-Seegmiller syndrome, kidney disease, chronic kidney disease, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency or a combination thereof. In some embodiments, the condition is gout. In some embodiments, the condition is chronic kidney disease. In some embodiments, the condition is heart failure. In some embodiments, the xanthine oxidase inhibitor is febuxostat or a pharmaceutically acceptable salt thereof. In some embodiments, the xanthine oxidase inhibitor is allopurinol or a pharmaceutically acceptable salt thereof.

In some embodiments, disclosed herein is a method of treating or preventing chronic kidney disease in a subject in need thereof, comprising administering to the subject: verinurad or a pharmaceutically acceptable salt thereof; allopurinol or a pharmaceutically acceptable salt thereof; and dapagliflozin. In some embodiments, disclosed herein is a method of treating or preventing heart failure in a subject in need thereof, comprising administering to the subject: verinurad or a pharmaceutically acceptable salt thereof; allopurinol or a pharmaceutically acceptable salt thereof; and dapagliflozin. In some embodiments, disclosed herein is a method of reducing serum uric acid levels in a subject who is currently being treated with dapagliflozin or a pharmaceutically acceptable salt thereof, comprising administering to the subject: verinurad or a pharmaceutically acceptable salt thereof; and a xanthine oxidase inhibitor. In some embodiments, the subject has diabetes mellitus. In some embodiments, the xanthine oxidase inhibitor is febuxostat or a pharmaceutically acceptable salt thereof. In some embodiments, the xanthine oxidase inhibitor is allopurinol or a pharmaceutically acceptable salt thereof.

In some embodiments, disclosed herein is a method of treating or preventing a condition associated with hyperuricemia in a subject who is currently being treated with dapagliflozin or a pharmaceutically acceptable salt thereof, comprising administering to the subject: verinurad or a pharmaceutically acceptable salt thereof; and a xanthine oxidase inhibitor. In some embodiments, the subject has diabetes mellitus. In some embodiments, the condition is gout, a recurrent gout attack, gouty arthritis, hypertension, a cardiovascular disease, coronary heart disease, heart failure, Lesch-Nyhan syndrome, Kelley-Seegmiller syndrome, kidney disease, chronic kidney disease, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency or a combination thereof. In some embodiments, the condition is gout. In some embodiments, the condition is chronic kidney disease. In some embodiments, the condition is heart failure. In some embodiments, the xanthine oxidase inhibitor is febuxostat or a pharmaceutically acceptable salt thereof. In some embodiments, the xanthine oxidase inhibitor is allopurinol or a pharmaceutically acceptable salt thereof.

In some embodiments, disclosed herein is a method of treating or preventing chronic kidney disease in a subject with diabetes mellitus who is currently being treated with dapagliflozin or a pharmaceutically acceptable salt thereof, comprising administering to the subject: verinurad or a pharmaceutically acceptable salt thereof; and allopurinol or a pharmaceutically acceptable salt thereof. In some embodiments, disclosed herein is a method of treating or preventing heart failure in a subject with diabetes mellitus who is currently being treated with dapagliflozin or a pharmaceutically acceptable salt thereof, comprising administering to the subject: verinurad or a pharmaceutically acceptable salt thereof; and allopurinol or a pharmaceutically acceptable salt thereof.

In some embodiments, the instant disclosure provides pharmaceutical compositions comprising an effective amount of a URAT1 inhibitor, an XOI, and an SGLT2 inhibitor. In some embodiments, disclosed herein is a pharmaceutical composition comprising: verinurad or a pharmaceutically acceptable salt thereof; a xanthine oxidase inhibitor; dapagliflozin; and a pharmaceutically acceptable excipient or carrier. In some embodiments, the xanthine oxidase inhibitor is febuxostat, or a pharmaceutically acceptable salt thereof. In some embodiments, the xanthine oxidase inhibitor is allopurinol, or a pharmaceutically acceptable salt thereof. In some embodiments, disclosed herein is a method of reducing serum uric acid levels in a subject in need thereof, comprising administering to the subject an effective amount of a pharmaceutical composition disclosed herein. In some embodiments, disclosed herein is a method of treating or preventing a condition associated with hyperuricemia in a subject in need thereof, comprising administering to the subject an effective amount of a pharmaceutical composition disclosed herein. In some embodiments, the condition is gout, a recurrent gout attack, gouty arthritis, hypertension, a cardiovascular disease, coronary heart disease, heart failure, Lesch-Nyhan syndrome, Kelley-Seegmiller syndrome, kidney disease, chronic kidney disease, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency or a combination thereof. In some embodiments, the condition is gout. In some embodiments, the condition is chronic kidney disease. In some embodiments, the condition is heart failure.

### DETAILED DESCRIPTION

### Definitions

The term “patient,” “subject” or “individual” are used interchangeably. As used herein, they refer to individuals suffering from a disorder, and the like. None of the terms require that the individual be under the care and/or supervision of a medical professional.

The terms “treat,” “treating” or “treatment,” and other grammatical equivalents as used herein, include alleviating, abating or ameliorating a disease or condition or one or more symptoms thereof, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular, or infusion), topical and rectal administration. Those of skill in the art are familiar with administration techniques that can be employed with the compounds, compositions, and methods described herein.

The terms “effective amount,” “therapeutically effective amount” or “pharmaceutically effective amount” as used herein, refer to a sufficient amount of at least one agent or compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount may differ from one individual to another. An appropriate “effective” amount in any individual case may be determined using techniques, such as a dose escalation study.

The term “pharmaceutically acceptable” as used herein, refers to a material, such as a carrier or diluent, which does not abrogate the biological activity or properties of the compounds described herein, and is relatively nontoxic, i.e., the material may be administered to an individual without causing undesirable biological effects or interacting in a deleterious manner with any of the components of the composition in which it is contained.

The term “pharmaceutically acceptable salt” as used herein, refers to salts that retain the biological effectiveness of the free acids and bases of the specified compound and that are not biologically or otherwise undesirable. Compounds described herein may possess acidic or basic groups and therefore may react with any of a number of inorganic or organic bases, and inorganic and organic acids, to form a pharmaceutically acceptable salt. These salts can be prepared in situ during the final isolation and purification of the compounds disclosed herein, or by separately reacting a purified compound in its free base form with a suitable organic or inorganic acid, and isolating the salt thus formed. In some embodiments, for example, verinurad is provided as a base addition salt, such as the sodium salt.

The term “pharmaceutical composition,” as used herein, refers to a composition comprising at least one active ingredient mixed with at least one pharmaceutically acceptable chemical component, such as, though not limited to carriers, stabilizers, diluents, dispersing agents, suspending agents, thickening agents, excipients and the like.

The terms “co-administration,” “administered in combination with” and their grammatical equivalents, as used herein, are meant to encompass administration of the active ingredients to a single individual, and are intended to include treatment regimens in which the agents are administered by the same or different route of administration, in the same or different pharmaceutical compositions, and at the same or different times. They include simultaneous administration in separate compositions, administration at different times in separate compositions, and/or administration in a composition in which all active ingredients are present.

### Therapeutic Methods

In some embodiments, the instant disclosure provides methods of reducing serum uric acid levels in a subject in need thereof, comprising administering to the subject: a URAT1 inhibitor; a xanthine oxidase inhibitor; and an SGLT2 inhibitor. In some embodiments, the URAT1 inhibitor is verinurad or a pharmaceutically acceptable salt thereof. In some embodiments, the URAT1 inhibitor is lesinurad or a pharmaceutically acceptable salt thereof. In some embodiments, the xanthine oxidase inhibitor is febuxostat or allopurinol, or a pharmaceutically acceptable salt thereof. In some embodiments, the xanthine oxidase inhibitor is febuxostat or a pharmaceutically acceptable salt thereof. In some embodiments, the xanthine oxidase inhibitor is febuxostat. In some embodiments, the xanthine oxidase inhibitor is allopurinol or a pharmaceutically acceptable salt thereof. In some embodiments, the xanthine oxidase inhibitor is allopurinol. In some embodiments, the SGLT2 inhibitor is canagliflozin, dapagliflozin, or empagliflozin, or a pharmaceutically acceptable salt thereof. In some embodiments, the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is allopurinol or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat; and the SGLT2 inhibitor is dapagliflozin. In some embodiments, the URAT1 inhibitor is verinurad or a pharmaceutically acceptable salt thereof; the xanthine oxidase inhibitor is allopurinol; and the SGLT2 inhibitor is dapagliflozin. In some embodiments, the subject is human. In some embodiments, the subject has diabetes mellitus. In some embodiments, the subject does not have diabetes mellitus.

In some embodiments, the instant disclosure provides a method of reducing serum uric acid levels in a subject who is currently being treated with an SGLT2 inhibitor, comprising administering to the subject: a URAT1 inhibitor; and a xanthine oxidase inhibitor. In some embodiments, the subject has diabetes mellitus. In some embodiments, the URAT1 inhibitor is verinurad or a pharmaceutically acceptable salt thereof. In some embodiments, the URAT1 inhibitor is lesinurad or a pharmaceutically acceptable salt thereof. In some embodiments, the xanthine oxidase inhibitor is febuxostat or allopurinol, or a pharmaceutically acceptable salt thereof. In some embodiments, the xanthine oxidase inhibitor is febuxostat or a pharmaceutically acceptable salt thereof. In some embodiments, the xanthine oxidase inhibitor is febuxostat. In some embodiments, the xanthine oxidase inhibitor is allopurinol or a pharmaceutically acceptable salt thereof. In some embodiments, the xanthine oxidase inhibitor is allopurinol. In some embodiments, the SGLT2 inhibitor is canagliflozin, dapagliflozin, or empagliflozin, or a pharmaceutically acceptable salt thereof. In some embodiments, the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is allopurinol or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat; and the SGLT2 inhibitor is dapagliflozin. In some embodiments, the URAT1 inhibitor is verinurad or a pharmaceutically acceptable salt thereof; the xanthine oxidase inhibitor is allopurinol; and the SGLT2 inhibitor is dapagliflozin. In some embodiments, the subject is human. In some embodiments, the instant disclosure provides a method of reducing serum uric acid levels in a subject who is currently being treated with dapagliflozin or a pharmaceutically acceptable salt thereof, comprising administering to the subject: verinurad or a pharmaceutically acceptable salt thereof; and a xanthine oxidase inhibitor. In some embodiments, the subject has diabetes mellitus. In some embodiments, the subject does not have diabetes mellitus.

### Active Ingredients

### URAT1 Inhibitors

Levels of urate anion in the blood are regulated in part by urate transporters. In particular instances, the urate transporter is URAT1. In some instances, single nucleotide polymorphisms of the gene which expresses URAT1 are significantly associated with increased or decreased reabsorption of uric acid by the kidneys, which contributes to hyperuricemia and hypouricemia, respectively.

Disclosed herein is the use of a URAT1 inhibitor in urate lowering combination therapy. In some embodiments, the URAT1 inhibitor is lesinurad, verinurad, or a pharmaceutically acceptable salt thereof. In particular embodiments, the URAT1 inhibitor is verinurad or a pharmaceutically acceptable salt thereof.

Verinurad is the generic name for 2-[3-(4-cyanonaphthalen-1-yl)pyridin-4-yl]sulfanyl-2-methylpropanoic acid, which has the following chemical structure:

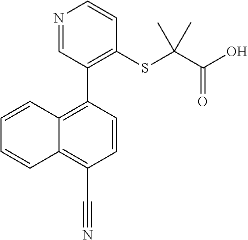 [SMILES: CC(C)(Sc1ccncc1-c1ccc(C#N)c2ccccc12)C(=O)O]

In some embodiments, a pharmaceutically acceptable salt of verinurad is the sodium salt of verinurad, i.e. sodium 2-[3-(4-cyanonaphthalen-1-yl)pyridin-4-yl]sulfanyl-2-methylpropanoate. In some embodiments, a crystalline solvate of verinurad is administered in a method or used in a composition disclosed herein. In some embodiments, a crystalline solvate of verinurad disclosed in U.S. Pat. No. 7,919,598 is administered in a method or used in a composition disclosed herein. In some embodiments, crystalline verinurad:(2S)-1,2-propanediol:H₂O (1:1:1) is administered in a method or used in a composition disclosed herein.

### Xanthine Oxidase Inhibitors

The oxidation of hypoxanthine to xanthine and further the oxidation of xanthine to uric acid can be catalyzed by xanthine oxidase. In some embodiments, disclosed herein is the use of a xanthine oxidase inhibitor (XOI) in urate lowering combination therapy. In some embodiments, the XOI is a purine analog, such as allopurinol, oxypurinol, or tisopurine. In other embodiments, the XOI is another molecule, such as febuxostat or topiroxostat. In some embodiments, the XOI is allopurinol or febuxostat. In some embodiments, the XOI is febuxostat. In embodiments, the XOI is allopurinol.

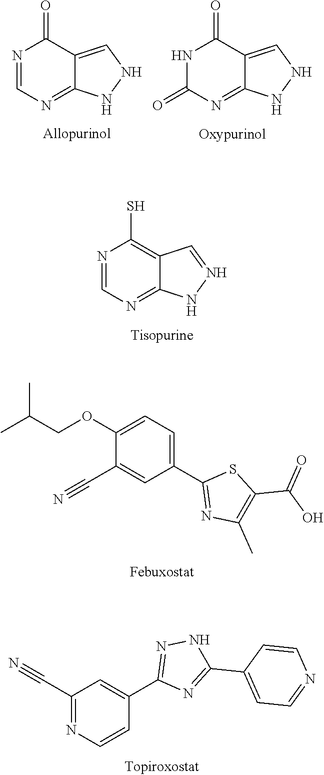

### Sodium-Glucose Transport Protein, Subtype 2 (SGLT2) Inhibitors

In some instances, glucose reabsorption in the kidney is regulated by a member of the sodium glucose cotransporter family, which are sodium-dependent glucose transport proteins. In some instances, glucose reabsorption in the kidney is regulated by sodium-glucose transport protein, subtype 2 (SGLT2). In some instances, inhibiting SGLT2 causes glycosuria. In particular instances, inhibiting SGLT2 causes glycosuria-induced alteration of uric acid transport activity in renal tubules. In some instances, inhibitors of SGLT2 lead to lower serum uric acid levels.

In some embodiments, disclosed herein is the use of a SGLT2 inhibitor in urate lowering combination therapy. In some embodiments, the SGLT2 inhibitor is a gliflozin. In some embodiments, the SGLT2 inhibitor is canagliflozin, dapagliflozin, empagliflozin, ertugliflozin, ipragliflozin, remogliflozin etabonate, sergliflozin etabonate, sotagliflozin, or tofogliflozin. In particular embodiments, the SGLT2 inhibitor is canagliflozin, dapagliflozin, or empagliflozin. In a particular embodiment, the SGLT2 inhibitor is dapagliflozin, which has the following chemical structure:

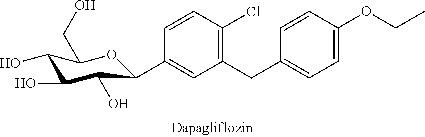

Conditions Associated with Hyperuricemia

Hyperuricemia is an abnormally high level of uric acid in the blood. Hyperuricemia may be asymptomatic. In certain instances, hyperuricemia is associated with at least one other disease or condition. In certain instances, hyperuricemia is associated with gout, a recurrent gout attack, gouty arthritis, hypertension, a cardiovascular disease, coronary heart disease, heart failure, Lesch-Nyhan syndrome, Kelley-Seegmiller syndrome, kidney disease, chronic kidney disease, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof. In some embodiments, the condition is gout. In some embodiments, the condition is chronic kidney disease. In some embodiments, the condition is heart failure.

In some embodiments, the instant disclosure provides methods of treating or preventing a condition associated with hyperuricemia in a subject in need thereof, comprising administering to the subject: a URAT1 inhibitor; a xanthine oxidase inhibitor; and an SGLT2 inhibitor. In some embodiments, the condition is gout, a recurrent gout attack, gouty arthritis, hypertension, a cardiovascular disease, coronary heart disease, heart failure, Lesch-Nyhan syndrome, Kelley-Seegmiller syndrome, kidney disease, chronic kidney disease, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency or a combination thereof. In some embodiments, the condition is gout. In some embodiments, the condition is chronic kidney disease. In some embodiments, the condition is heart failure. In some embodiments, the URAT1 inhibitor is verinurad or a pharmaceutically acceptable salt thereof. In some embodiments, the URAT1 inhibitor is lesinurad or a pharmaceutically acceptable salt thereof. In some embodiments, the xanthine oxidase inhibitor is febuxostat or allopurinol, or a pharmaceutically acceptable salt thereof. In some embodiments, the xanthine oxidase inhibitor is febuxostat or a pharmaceutically acceptable salt thereof. In some embodiments, the xanthine oxidase inhibitor is febuxostat. In some embodiments, the xanthine oxidase inhibitor is allopurinol or a pharmaceutically acceptable salt thereof. In some embodiments, the xanthine oxidase inhibitor is allopurinol. In some embodiments, the SGLT2 inhibitor is canagliflozin, dapagliflozin, or empagliflozin, or a pharmaceutically acceptable salt thereof. In some embodiments, the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is allopurinol or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat; and the SGLT2 inhibitor is dapagliflozin. In some embodiments, the URAT1 inhibitor is verinurad or a pharmaceutically acceptable salt thereof; the xanthine oxidase inhibitor is allopurinol; and the SGLT2 inhibitor is dapagliflozin. In some embodiments, the subject is human.

In some embodiments, the instant disclosure provides a method of treating or preventing a condition associated with hyperuricemia in a subject who is currently being treated with an SGLT2 inhibitor, comprising administering to the subject: a URAT1 inhibitor; and a xanthine oxidase inhibitor. In some embodiments, the condition is gout, a recurrent gout attack, gouty arthritis, hypertension, a cardiovascular disease, coronary heart disease, heart failure, Lesch-Nyhan syndrome, Kelley-Seegmiller syndrome, kidney disease, chronic kidney disease, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency or a combination thereof. In some embodiments, the condition is gout. In some embodiments, the condition is chronic kidney disease. In some embodiments, the condition is heart failure. In some embodiments, the subject has diabetes mellitus. In some embodiments, the URAT1 inhibitor is verinurad or a pharmaceutically acceptable salt thereof. In some embodiments, the URAT1 inhibitor is lesinurad or a pharmaceutically acceptable salt thereof. In some embodiments, the xanthine oxidase inhibitor is febuxostat or allopurinol, or a pharmaceutically acceptable salt thereof. In some embodiments, the xanthine oxidase inhibitor is febuxostat or a pharmaceutically acceptable salt thereof. In some embodiments, the xanthine oxidase inhibitor is febuxostat. In some embodiments, the xanthine oxidase inhibitor is allopurinol or a pharmaceutically acceptable salt thereof. In some embodiments, the xanthine oxidase inhibitor is allopurinol. In some embodiments, the SGLT2 inhibitor is canagliflozin, dapagliflozin, or empagliflozin, or a pharmaceutically acceptable salt thereof. In some embodiments, the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is allopurinol or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat; and the SGLT2 inhibitor is dapagliflozin. In some embodiments, the URAT1 inhibitor is verinurad or a pharmaceutically acceptable salt thereof; the xanthine oxidase inhibitor is allopurinol; and the SGLT2 inhibitor is dapagliflozin. In some embodiments, the subject is human.

In some embodiments, the instant disclosure provides a method of treating or preventing a condition associated with hyperuricemia in a subject who is currently being treated with dapagliflozin or a pharmaceutically acceptable salt thereof, comprising administering to the subject: verinurad or a pharmaceutically acceptable salt thereof; and allopurinol or a pharmaceutically acceptable salt thereof. In some embodiments, the subject has diabetes mellitus. In some embodiments, the instant disclosure provides a method of treating or preventing a condition associated with hyperuricemia in a subject who is currently being treated with dapagliflozin or a pharmaceutically acceptable salt thereof, comprising administering to the subject: verinurad or a pharmaceutically acceptable salt thereof; and febuxostat or a pharmaceutically acceptable salt thereof. In some embodiments, the subject has diabetes mellitus.

### Kidney Disease

In some instances, disclosed herein is a method of treating or preventing kidney disease in a subject in need thereof, comprising administering to the subject: a URAT1 inhibitor; a xanthine oxidase inhibitor; and an SGLT2 inhibitor. In some embodiments, the kidney disease is acute kidney disease, acute kidney failure, chronic kidney disease, chronic kidney failure, or diabetic kidney disease. In some embodiments, the kidney disease is chronic kidney disease. In some embodiments, the kidney disease is diabetic kidney disease. In some embodiments, the URAT1 inhibitor is verinurad or a pharmaceutically acceptable salt thereof. In some embodiments, the URAT1 inhibitor is lesinurad or a pharmaceutically acceptable salt thereof. In some embodiments, the xanthine oxidase inhibitor is febuxostat or allopurinol, or a pharmaceutically acceptable salt thereof. In some embodiments, the xanthine oxidase inhibitor is febuxostat or a pharmaceutically acceptable salt thereof. In some embodiments, the xanthine oxidase inhibitor is febuxostat. In some embodiments, the xanthine oxidase inhibitor is allopurinol or a pharmaceutically acceptable salt thereof. In some embodiments, the xanthine oxidase inhibitor is allopurinol. In some embodiments, the SGLT2 inhibitor is canagliflozin, dapagliflozin, or empagliflozin, or a pharmaceutically acceptable salt thereof. In some embodiments, the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is allopurinol or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat; and the SGLT2 inhibitor is dapagliflozin. In some embodiments, the URAT1 inhibitor is verinurad or a pharmaceutically acceptable salt thereof; the xanthine oxidase inhibitor is allopurinol; and the SGLT2 inhibitor is dapagliflozin. In some embodiments, the subject is human.

In some embodiments, the instant disclosure provides a method of treating or preventing chronic kidney disease in a subject with diabetes mellitus who is currently being treated with dapagliflozin or a pharmaceutically acceptable salt thereof, comprising administering to the subject verinurad or a pharmaceutically acceptable salt thereof; and allopurinol or a pharmaceutically acceptable salt thereof. In some embodiments, the instant disclosure provides a method of treating or preventing chronic kidney disease in a subject with diabetes mellitus who is currently being treated with dapagliflozin or a pharmaceutically acceptable salt thereof, comprising administering to the subject verinurad or a pharmaceutically acceptable salt thereof; and febuxostat or a pharmaceutically acceptable salt thereof.

### Cardiovascular Disease

In some instances, disclosed herein is method of treating or preventing cardiovascular disease in a subject in need thereof, comprising administering to the subject: a URAT1 inhibitor; a xanthine oxidase inhibitor; and an SGLT2 inhibitor. In some embodiments, the cardiovascular disease is coronary heart disease or heart failure. In some embodiments, the cardiovascular disease is heart failure. In some embodiments, the URAT1 inhibitor is verinurad or a pharmaceutically acceptable salt thereof. In some embodiments, the URAT1 inhibitor is lesinurad or a pharmaceutically acceptable salt thereof. In some embodiments, the xanthine oxidase inhibitor is febuxostat or allopurinol, or a pharmaceutically acceptable salt thereof. In some embodiments, the xanthine oxidase inhibitor is febuxostat or a pharmaceutically acceptable salt thereof. In some embodiments, the xanthine oxidase inhibitor is febuxostat. In some embodiments, the xanthine oxidase inhibitor is allopurinol or a pharmaceutically acceptable salt thereof. In some embodiments, the xanthine oxidase inhibitor is allopurinol. In some embodiments, the SGLT2 inhibitor is canagliflozin, dapagliflozin, or empagliflozin, or a pharmaceutically acceptable salt thereof. In some embodiments, the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is allopurinol or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat; and the SGLT2 inhibitor is dapagliflozin. In some embodiments, the URAT1 inhibitor is verinurad or a pharmaceutically acceptable salt thereof; the xanthine oxidase inhibitor is allopurinol; and the SGLT2 inhibitor is dapagliflozin. In some embodiments, the subject is human.

In some embodiments, the instant disclosure provides a method of treating or preventing heart failure in a subject with diabetes mellitus who is currently being treated with dapagliflozin or a pharmaceutically acceptable salt thereof, comprising administering to the subject verinurad or a pharmaceutically acceptable salt thereof; and allopurinol or a pharmaceutically acceptable salt thereof. In some embodiments, the instant disclosure provides a method of treating or preventing heart failure in a subject with diabetes mellitus who is currently being treated with dapagliflozin or a pharmaceutically acceptable salt thereof, comprising administering to the subject verinurad or a pharmaceutically acceptable salt thereof; and febuxostat or a pharmaceutically acceptable salt thereof.

### Gout

In another aspect, disclosed herein is method of treating or preventing gout in a subject in need thereof, comprising administering to the subject: a URAT1 inhibitor; a xanthine oxidase inhibitor; and an SGLT2 inhibitor. In some embodiments, the URAT1 inhibitor is verinurad or a pharmaceutically acceptable salt thereof. In some embodiments, the URAT1 inhibitor is lesinurad or a pharmaceutically acceptable salt thereof. In some embodiments, the xanthine oxidase inhibitor is febuxostat or allopurinol, or a pharmaceutically acceptable salt thereof. In some embodiments, the xanthine oxidase inhibitor is febuxostat or a pharmaceutically acceptable salt thereof. In some embodiments, the xanthine oxidase inhibitor is febuxostat. In some embodiments, the xanthine oxidase inhibitor is allopurinol or a pharmaceutically acceptable salt thereof. In some embodiments, the xanthine oxidase inhibitor is allopurinol. In some embodiments, the SGLT2 inhibitor is canagliflozin, dapagliflozin, or empagliflozin, or a pharmaceutically acceptable salt thereof. In some embodiments, the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is allopurinol or a pharmaceutically acceptable salt thereof; and the SGLT2 inhibitor is dapagliflozin or a pharmaceutically acceptable salt thereof. In some embodiments, the URAT1 inhibitor is verinurad or a pharmaceutically acceptable salt thereof; the xanthine oxidase inhibitor is febuxostat; and the SGLT2 inhibitor is dapagliflozin. In some embodiments, the URAT1 inhibitor is verinurad or a pharmaceutically acceptable salt thereof; the xanthine oxidase inhibitor is allopurinol; and the SGLT2 inhibitor is dapagliflozin. In some embodiments, the subject is human.

In some embodiments, the instant disclosure provides a method of treating or preventing gout in a subject with diabetes mellitus who is currently being treated with dapagliflozin or a pharmaceutically acceptable salt thereof, comprising administering to the subject verinurad or a pharmaceutically acceptable salt thereof; and allopurinol or a pharmaceutically acceptable salt thereof. In some embodiments, the instant disclosure provides a method of treating or preventing gout in a subject with diabetes mellitus who is currently being treated with dapagliflozin or a pharmaceutically acceptable salt thereof, comprising administering to the subject verinurad or a pharmaceutically acceptable salt thereof; and febuxostat or a pharmaceutically acceptable salt thereof.

### Dosing

The amount of active ingredient(s) administered in a method disclosed herein will firstly be dependent on the patient being treated. The daily dosage will normally be determined by the prescribing physician with the dosage generally varying according to the age, sex, diet, weight, general health, genetics, response of the individual, the severity of the individual's symptoms, the precise indication or condition being treated, the severity of the indication or condition being treated, time of administration, route of administration, the disposition of the composition, rate of excretion, drug combination, and the discretion of the prescribing physician. The amount and frequency of administration of the compounds described herein, and if applicable other therapeutic agents and/or therapies, will be regulated according to the judgment of the attending clinician (physician) considering such factors as described above. Thus, the amount of pharmaceutical composition to be administered may vary widely.

In some embodiments, the methods comprise administering between 1 mg and 20 mg of verinurad or a pharmaceutically acceptable salt thereof; between 100 mg and 600 mg of allopurinol; and between about 1 mg and 20 mg of dapagliflozin. In some embodiments, the methods comprise administering between 5 mg and 15 mg of verinurad or a pharmaceutically acceptable salt thereof; between 100 mg and 400 mg of allopurinol; and between about 5 mg and 15 mg of dapagliflozin. In some embodiments, the methods comprise administering about 9 mg of verinurad or a pharmaceutically acceptable salt thereof; about 100 mg of allopurinol; and about 5 mg of dapagliflozin. In some embodiments, the methods comprise administering about 9 mg of verinurad or a pharmaceutically acceptable salt thereof; about 200 mg of allopurinol; and about 5 mg of dapagliflozin. In some embodiments, the methods comprise administering about 9 mg of verinurad or a pharmaceutically acceptable salt thereof; about 300 mg of allopurinol; and about 5 mg of dapagliflozin. In some embodiments, the methods comprise administering about 9 mg of verinurad or a pharmaceutically acceptable salt thereof; about 400 mg of allopurinol; and about 5 mg of dapagliflozin. In some embodiments, the methods comprise administering about 9 mg of verinurad or a pharmaceutically acceptable salt thereof; about 100 mg of allopurinol; and about 10 mg of dapagliflozin. In some embodiments, the methods comprise administering about 9 mg of verinurad or a pharmaceutically acceptable salt thereof; about 200 mg of allopurinol; and about10 mg of dapagliflozin. In some embodiments, the methods comprise administering about 9 mg of verinurad or a pharmaceutically acceptable salt thereof; about 300 mg of allopurinol; and about 10 mg of dapagliflozin. In some embodiments, the methods comprise administering about 9 mg of verinurad or a pharmaceutically acceptable salt thereof; about 400 mg of allopurinol; and about 10 mg of dapagliflozin. In some embodiments, the methods comprise administering about 9 mg of verinurad or a pharmaceutically acceptable salt thereof; about 300 mg of allopurinol; and about 5 mg of dapagliflozin. In some embodiments, the methods comprise administering about 2 mg of verinurad or a pharmaceutically acceptable salt thereof; about 300 mg of allopurinol; and about 5 mg of dapagliflozin. In some embodiments, the methods comprise administering about 3 mg of verinurad or a pharmaceutically acceptable salt thereof; about 300 mg of allopurinol; and about 5 mg of dapagliflozin. In some embodiments, the methods comprise administering about 4 mg of verinurad or a pharmaceutically acceptable salt thereof; about 300 mg of allopurinol; and about 5 mg of dapagliflozin. In some embodiments, the methods comprise administering about 5 mg of verinurad or a pharmaceutically acceptable salt thereof; about 300 mg of allopurinol; and about 5 mg of dapagliflozin. In some embodiments, the methods comprise administering about 6 mg of verinurad or a pharmaceutically acceptable salt thereof; about 300 mg of allopurinol; and about 5 mg of dapagliflozin. In some embodiments, the methods comprise administering about 7 mg of verinurad or a pharmaceutically acceptable salt thereof; about 300 mg of allopurinol; and about 5 mg of dapagliflozin. In some embodiments, the methods comprise administering about 7.5 mg of verinurad or a pharmaceutically acceptable salt thereof; about 300 mg of allopurinol; and about 5 mg of dapagliflozin. In some embodiments, the methods comprise administering about 8 mg of verinurad or a pharmaceutically acceptable salt thereof; about 300 mg of allopurinol; and about 5 mg of dapagliflozin. In some embodiments, the methods comprise administering about 9 mg of verinurad or a pharmaceutically acceptable salt thereof; about 300 mg of allopurinol; and about 5 mg of dapagliflozin. In some embodiments, the methods comprise administering about 10 mg of verinurad or a pharmaceutically acceptable salt thereof; about 300 mg of allopurinol; and about 5 mg of dapagliflozin. In some embodiments, the methods comprise administering about 11 mg of verinurad or a pharmaceutically acceptable salt thereof; about 300 mg of allopurinol; and about 5 mg of dapagliflozin. In some embodiments, the methods comprise administering about 12 mg of verinurad or a pharmaceutically acceptable salt thereof; about 300 mg of allopurinol; and about 5 mg of dapagliflozin. In some embodiments, the methods comprise administering about 13 mg of verinurad or a pharmaceutically acceptable salt thereof; about 300 mg of allopurinol; and about 5 mg of dapagliflozin. In some embodiments, the methods comprise administering about 14 mg of verinurad or a pharmaceutically acceptable salt thereof; about 300 mg of allopurinol; and about 5 mg of dapagliflozin. In some embodiments, the methods comprise administering about 15 mg of verinurad or a pharmaceutically acceptable salt thereof; about 300 mg of allopurinol; and about 5 mg of dapagliflozin. In some embodiments, the methods comprise administering about 2 mg of verinurad or a pharmaceutically acceptable salt thereof; about 300 mg of allopurinol; and about 10 mg of dapagliflozin. In some embodiments, the methods comprise administering about 3 mg of verinurad or a pharmaceutically acceptable salt thereof; about 300 mg of allopurinol; and about 10 mg of dapagliflozin. In some embodiments, the methods comprise administering about 4 mg of verinurad or a pharmaceutically acceptable salt thereof; about 300 mg of allopurinol; and about 10 mg of dapagliflozin. In some embodiments, the methods comprise administering about 5 mg of verinurad or a pharmaceutically acceptable salt thereof; about 300 mg of allopurinol; and about 10 mg of dapagliflozin. In some embodiments, the methods comprise administering about 6 mg of verinurad or a pharmaceutically acceptable salt thereof; about 300 mg of allopurinol; and about 10 mg of dapagliflozin. In some embodiments, the methods comprise administering about 7 mg of verinurad or a pharmaceutically acceptable salt thereof; about 300 mg of allopurinol; and about 10 mg of dapagliflozin. In some embodiments, the methods comprise administering about 7.5 mg of verinurad or a pharmaceutically acceptable salt thereof; about 300 mg of allopurinol; and about 10 mg of dapagliflozin. In some embodiments, the methods comprise administering about 8 mg of verinurad or a pharmaceutically acceptable salt thereof; about 300 mg of allopurinol; and about 10 mg of dapagliflozin. In some embodiments, the methods comprise administering about 9 mg of verinurad or a pharmaceutically acceptable salt thereof; about 300 mg of allopurinol; and about 10 mg of dapagliflozin. In some embodiments, the methods comprise administering about 10 mg of verinurad or a pharmaceutically acceptable salt thereof; about 300 mg of allopurinol; and about 10 mg of dapagliflozin. In some embodiments, the methods comprise administering about 11 mg of verinurad or a pharmaceutically acceptable salt thereof; about 300 mg of allopurinol; and about 10 mg of dapagliflozin. In some embodiments, the methods comprise administering about 12 mg of verinurad or a pharmaceutically acceptable salt thereof; about 300 mg of allopurinol; and about 10 mg of dapagliflozin. In some embodiments, the methods comprise administering about 13 mg of verinurad or a pharmaceutically acceptable salt thereof; about 300 mg of allopurinol; and about 10 mg of dapagliflozin. In some embodiments, the methods comprise administering about 14 mg of verinurad or a pharmaceutically acceptable salt thereof; about 300 mg of allopurinol; and about 10 mg of dapagliflozin. In some embodiments, the methods comprise administering about 15 mg of verinurad or a pharmaceutically acceptable salt thereof; about 300 mg of allopurinol; and about 10 mg of dapagliflozin.

In some embodiments, the methods comprise administering between 1 mg and 20 mg of verinurad or a pharmaceutically acceptable salt thereof per day; between 100 mg and 600 mg of allopurinol per day; and between about 1 mg and 20 mg of dapagliflozin per day. In some embodiments, the methods comprise administering between 5 mg and 15 mg of verinurad or a pharmaceutically acceptable salt thereof per day; between 100 mg and 400 mg of allopurinol per day; and between about 5 mg and 15 mg of dapagliflozin per day. In some embodiments, the methods comprise administering about 9 mg of verinurad or a pharmaceutically acceptable salt thereof per day; about 100 mg of allopurinol per day; and about 5 mg of dapagliflozin per day. In some embodiments, the methods comprise administering about 9 mg of verinurad or a pharmaceutically acceptable salt thereof per day; about 200 mg of allopurinol per day; and about 5 mg of dapagliflozin per day. In some embodiments, the methods comprise administering about 9 mg of verinurad or a pharmaceutically acceptable salt thereof per day; about 300 mg of allopurinol per day; and about 5 mg of dapagliflozin per day. In some embodiments, the methods comprise administering about 9 mg of verinurad or a pharmaceutically acceptable salt thereof per day; about 400 mg of allopurinol per day; and about 5 mg of dapagliflozin per day. In some embodiments, the methods comprise administering about 9 mg of verinurad or a pharmaceutically acceptable salt thereof per day; about 100 mg of allopurinol per day; and about 10 mg of dapagliflozin per day. In some embodiments, the methods comprise administering about 9 mg of verinurad or a pharmaceutically acceptable salt thereof per day; about 200 mg of allopurinol per day; and about10 mg of dapagliflozin per day. In some embodiments, the methods comprise administering about 9 mg of verinurad or a pharmaceutically acceptable salt thereof per day; about 300 mg of allopurinol per day; and about 10 mg of dapagliflozin per day. In some embodiments, the methods comprise administering about 9 mg of verinurad or a pharmaceutically acceptable salt thereof per day; about 400 mg of allopurinol per day; and about 10 mg of dapagliflozin per day. In some embodiments, the methods comprise administering about 9 mg of verinurad or a pharmaceutically acceptable salt thereof per day; about 300 mg of allopurinol per day; and about 5 mg of dapagliflozin per day. In some embodiments, the methods comprise administering about 2 mg of verinurad or a pharmaceutically acceptable salt thereof per day; about 300 mg of allopurinol per day; and about 5 mg of dapagliflozin per day. In some embodiments, the methods comprise administering about 3 mg of verinurad or a pharmaceutically acceptable salt thereof per day; about 300 mg of allopurinol per day; and about 5 mg of dapagliflozin per day. In some embodiments, the methods comprise administering about 4 mg of verinurad or a pharmaceutically acceptable salt thereof per day; about 300 mg of allopurinol per day; and about 5 mg of dapagliflozin per day. In some embodiments, the methods comprise administering about 5 mg of verinurad or a pharmaceutically acceptable salt thereof per day; about 300 mg of allopurinol per day; and about 5 mg of dapagliflozin per day. In some embodiments, the methods comprise administering about 6 mg of verinurad or a pharmaceutically acceptable salt thereof per day; about 300 mg of allopurinol per day; and about 5 mg of dapagliflozin per day. In some embodiments, the methods comprise administering about 7 mg of verinurad or a pharmaceutically acceptable salt thereof per day; about 300 mg of allopurinol per day; and about 5 mg of dapagliflozin per day. In some embodiments, the methods comprise administering about 7.5 mg of verinurad or a pharmaceutically acceptable salt thereof per day; about 300 mg of allopurinol per day; and about 5 mg of dapagliflozin per day. In some embodiments, the methods comprise administering about 8 mg of verinurad or a pharmaceutically acceptable salt thereof per day; about 300 mg of allopurinol per day; and about 5 mg of dapagliflozin per day. In some embodiments, the methods comprise administering about 9 mg of verinurad or a pharmaceutically acceptable salt thereof per day; about 300 mg of allopurinol per day; and about 5 mg of dapagliflozin per day. In some embodiments, the methods comprise administering about 10 mg of verinurad or a pharmaceutically acceptable salt thereof per day; about 300 mg of allopurinol per day; and about 5 mg of dapagliflozin per day. In some embodiments, the methods comprise administering about 11 mg of verinurad or a pharmaceutically acceptable salt thereof per day; about 300 mg of allopurinol per day; and about 5 mg of dapagliflozin per day. In some embodiments, the methods comprise administering about 12 mg of verinurad or a pharmaceutically acceptable salt thereof per day; about 300 mg of allopurinol per day; and about 5 mg of dapagliflozin per day. In some embodiments, the methods comprise administering about 13 mg of verinurad or a pharmaceutically acceptable salt thereof per day; about 300 mg of allopurinol per day; and about 5 mg of dapagliflozin per day. In some embodiments, the methods comprise administering about 14 mg of verinurad or a pharmaceutically acceptable salt thereof per day; about 300 mg of allopurinol per day; and about 5 mg of dapagliflozin per day. In some embodiments, the methods comprise administering about 15 mg of verinurad or a pharmaceutically acceptable salt thereof per day; about 300 mg of allopurinol per day; and about 5 mg of dapagliflozin per day. In some embodiments, the methods comprise administering about 2 mg of verinurad or a pharmaceutically acceptable salt thereof per day; about 300 mg of allopurinol per day; and about 10 mg of dapagliflozin per day. In some embodiments, the methods comprise administering about 3 mg of verinurad or a pharmaceutically acceptable salt thereof per day; about 300 mg of allopurinol per day; and about 10 mg of dapagliflozin per day. In some embodiments, the methods comprise administering about 4 mg of verinurad or a pharmaceutically acceptable salt thereof per day; about 300 mg of allopurinol per day; and about 10 mg of dapagliflozin per day. In some embodiments, the methods comprise administering about 5 mg of verinurad or a pharmaceutically acceptable salt thereof per day; about 300 mg of allopurinol per day; and about 10 mg of dapagliflozin per day. In some embodiments, the methods comprise administering about 6 mg of verinurad or a pharmaceutically acceptable salt thereof per day; about 300 mg of allopurinol per day; and about 10 mg of dapagliflozin per day. In some embodiments, the methods comprise administering about 7 mg of verinurad or a pharmaceutically acceptable salt thereof per day; about 300 mg of allopurinol per day; and about 10 mg of dapagliflozin per day. In some embodiments, the methods comprise administering about 7.5 mg of verinurad or a pharmaceutically acceptable salt thereof per day; about 300 mg of allopurinol per day; and about 10 mg of dapagliflozin per day. In some embodiments, the methods comprise administering about 8 mg of verinurad or a pharmaceutically acceptable salt thereof per day; about 300 mg of allopurinol per day; and about 10 mg of dapagliflozin per day. In some embodiments, the methods comprise administering about 9 mg of verinurad or a pharmaceutically acceptable salt thereof per day; about 300 mg of allopurinol per day; and about 10 mg of dapagliflozin per day. In some embodiments, the methods comprise administering about 10 mg of verinurad or a pharmaceutically acceptable salt thereof per day; about 300 mg of allopurinol per day; and about 10 mg of dapagliflozin per day. In some embodiments, the methods comprise administering about 11 mg of verinurad or a pharmaceutically acceptable salt thereof per day; about 300 mg of allopurinol per day; and about 10 mg of dapagliflozin per day. In some embodiments, the methods comprise administering about 12 mg of verinurad or a pharmaceutically acceptable salt thereof per day; about 300 mg of allopurinol per day; and about 10 mg of dapagliflozin per day. In some embodiments, the methods comprise administering about 13 mg of verinurad or a pharmaceutically acceptable salt thereof per day; about 300 mg of allopurinol per day; and about 10 mg of dapagliflozin per day. In some embodiments, the methods comprise administering about 14 mg of verinurad or a pharmaceutically acceptable salt thereof per day; about 300 mg of allopurinol per day; and about 10 mg of dapagliflozin per day. In some embodiments, the methods comprise administering about 15 mg of verinurad or a pharmaceutically acceptable salt thereof per day; about 300 mg of allopurinol per day; and about 10 mg of dapagliflozin per day.

In some embodiments, the methods described herein comprise administering between 1 mg and 20 mg of verinurad or a pharmaceutically acceptable salt thereof; between 10 mg and 200 mg of febuxostat; and between about 1 mg and 20 mg of dapagliflozin. In some embodiments, the methods comprise administering between 5 mg and 15 mg of verinurad or a pharmaceutically acceptable salt thereof; between 40 mg and 100 mg of febuxostat; and between about 5 mg and 15 mg of dapagliflozin. In some embodiments, the methods comprise administering about 9 mg of verinurad or a pharmaceutically acceptable salt thereof; about 40 mg of febuxostat; and about 5 mg of dapagliflozin. In some embodiments, the methods comprise administering about 9 mg of verinurad or a pharmaceutically acceptable salt thereof; about 80 mg of febuxostat; and about 5 mg of dapagliflozin. In some embodiments, the methods comprise administering about 9 mg of verinurad or a pharmaceutically acceptable salt thereof; about 40 mg of febuxostat; and about 10 mg of dapagliflozin. In some embodiments, the methods comprise administering about 9 mg of verinurad or a pharmaceutically acceptable salt thereof; about 80 mg of febuxostat; and about 10 mg of dapagliflozin. In some embodiments, the methods comprise administering about 2 mg of verinurad or a pharmaceutically acceptable salt thereof; about 80 mg of febuxostat; and about 5 mg of dapagliflozin. In some embodiments, the methods comprise administering about 3 mg of verinurad or a pharmaceutically acceptable salt thereof; about 80 mg of febuxostat; and about 5 mg of dapagliflozin. In some embodiments, the methods comprise administering about 4 mg of verinurad or a pharmaceutically acceptable salt thereof; about 80 mg of febuxostat; and about 5 mg of dapagliflozin. In some embodiments, the methods comprise administering about 5 mg of verinurad or a pharmaceutically acceptable salt thereof; about 80 mg of febuxostat; and about 5 mg of dapagliflozin. In some embodiments, the methods comprise administering about 6 mg of verinurad or a pharmaceutically acceptable salt thereof; about 80 mg of febuxostat; and about 5 mg of dapagliflozin. In some embodiments, the methods comprise administering about 7 mg of verinurad or a pharmaceutically acceptable salt thereof; about 80 mg of febuxostat; and about 5 mg of dapagliflozin. In some embodiments, the methods comprise administering about 7.5 mg of verinurad or a pharmaceutically acceptable salt thereof; about 80 mg of febuxostat; and about 5 mg of dapagliflozin. In some embodiments, the methods comprise administering about 8 mg of verinurad or a pharmaceutically acceptable salt thereof; about 80 mg of febuxostat; and about 5 mg of dapagliflozin. In some embodiments, the methods comprise administering about 9 mg of verinurad or a pharmaceutically acceptable salt thereof; about 80 mg of febuxostat; and about 5 mg of dapagliflozin. In some embodiments, the methods comprise administering about 10 mg of verinurad or a pharmaceutically acceptable salt thereof; about 80 mg of febuxostat; and about 5 mg of dapagliflozin. In some embodiments, the methods comprise administering about 11 mg of verinurad or a pharmaceutically acceptable salt thereof; about 80 mg of febuxostat; and about 5 mg of dapagliflozin. In some embodiments, the methods comprise administering about 12 mg of verinurad or a pharmaceutically acceptable salt thereof; about 80 mg of febuxostat; and about 5 mg of dapagliflozin. In some embodiments, the methods comprise administering about 13 mg of verinurad or a pharmaceutically acceptable salt thereof; about 80 mg of febuxostat; and about 5 mg of dapagliflozin. In some embodiments, the methods comprise administering about 14 mg of verinurad or a pharmaceutically acceptable salt thereof; about 80 mg of febuxostat; and about 5 mg of dapagliflozin. In some embodiments, the methods comprise administering about 15 mg of verinurad or a pharmaceutically acceptable salt thereof; about 80 mg of febuxostat; and about 5 mg of dapagliflozin. In some embodiments, the methods comprise administering about 2 mg of verinurad or a pharmaceutically acceptable salt thereof; about 80 mg of febuxostat; and about 10 mg of dapagliflozin. In some embodiments, the methods comprise administering about 3 mg of verinurad or a pharmaceutically acceptable salt thereof; about 80 mg of febuxostat; and about 10 mg of dapagliflozin. In some embodiments, the methods comprise administering about 4 mg of verinurad or a pharmaceutically acceptable salt thereof; about 80 mg of febuxostat; and about 10 mg of dapagliflozin. In some embodiments, the methods comprise administering about 5 mg of verinurad or a pharmaceutically acceptable salt thereof; about 80 mg of febuxostat; and about 10 mg of dapagliflozin. In some embodiments, the methods comprise administering about 6 mg of verinurad or a pharmaceutically acceptable salt thereof; about 80 mg of febuxostat; and about 10 mg of dapagliflozin. In some embodiments, the methods comprise administering about 7 mg of verinurad or a pharmaceutically acceptable salt thereof; about 80 mg of febuxostat; and about 10 mg of dapagliflozin. In some embodiments, the methods comprise administering about 7.5 mg of verinurad or a pharmaceutically acceptable salt thereof; about 80 mg of febuxostat; and about 10 mg of dapagliflozin. In some embodiments, the methods comprise administering about 8 mg of verinurad or a pharmaceutically acceptable salt thereof; about 80 mg of febuxostat; and about 10 mg of dapagliflozin. In some embodiments, the methods comprise administering about 9 mg of verinurad or a pharmaceutically acceptable salt thereof; about 80 mg of febuxostat; and about 10 mg of dapagliflozin. In some embodiments, the methods comprise administering about 10 mg of verinurad or a pharmaceutically acceptable salt thereof; about 80 mg of febuxostat; and about 10 mg of dapagliflozin. In some embodiments, the methods comprise administering about 11 mg of verinurad or a pharmaceutically acceptable salt thereof; about 80 mg of febuxostat; and about 10 mg of dapagliflozin. In some embodiments, the methods comprise administering about 12 mg of verinurad or a pharmaceutically acceptable salt thereof; about 80 mg of febuxostat; and about 10 mg of dapagliflozin. In some embodiments, the methods comprise administering about 13 mg of verinurad or a pharmaceutically acceptable salt thereof; about 80 mg of febuxostat; and about 10 mg of dapagliflozin. In some embodiments, the methods comprise administering about 14 mg of verinurad or a pharmaceutically acceptable salt thereof; about 80 mg of febuxostat; and about 10 mg of dapagliflozin. In some embodiments, the methods comprise administering about 15 mg of verinurad or a pharmaceutically acceptable salt thereof; about 80 mg of febuxostat; and about 10 mg of dapagliflozin.

In some embodiments, the methods described herein comprise administering between 1 mg and 20 mg of verinurad or a pharmaceutically acceptable salt thereof per day; between 10 mg and 200 mg of febuxostat per day; and between about 1 mg and 20 mg of dapagliflozin per day. In some embodiments, the methods comprise administering between 5 mg and 15 mg of verinurad or a pharmaceutically acceptable salt thereof per day; between 40 mg and 100 mg of febuxostat per day; and between about 5 mg and 15 mg of dapagliflozin per day. In some embodiments, the methods comprise administering about 9 mg of verinurad or a pharmaceutically acceptable salt thereof per day; about 40 mg of febuxostat per day; and about 5 mg of dapagliflozin per day. In some embodiments, the methods comprise administering about 9 mg of verinurad or a pharmaceutically acceptable salt thereof per day; about 80 mg of febuxostat per day; and about 5 mg of dapagliflozin per day. In some embodiments, the methods comprise administering about 9 mg of verinurad or a pharmaceutically acceptable salt thereof per day; about 40 mg of febuxostat per day; and about 10 mg of dapagliflozin per day. In some embodiments, the methods comprise administering about 9 mg of verinurad or a pharmaceutically acceptable salt thereof per day; about 80 mg of febuxostat per day; and about 10 mg of dapagliflozin per day. In some embodiments, the methods comprise administering about 2 mg of verinurad or a pharmaceutically acceptable salt thereof per day; about 80 mg of febuxostat per day; and about 5 mg of dapagliflozin per day. In some embodiments, the methods comprise administering about 3 mg of verinurad or a pharmaceutically acceptable salt thereof per day; about 80 mg of febuxostat per day; and about 5 mg of dapagliflozin per day. In some embodiments, the methods comprise administering about 4 mg of verinurad or a pharmaceutically acceptable salt thereof per day; about 80 mg of febuxostat per day; and about 5 mg of dapagliflozin per day. In some embodiments, the methods comprise administering about 5 mg of verinurad or a pharmaceutically acceptable salt thereof per day; about 80 mg of febuxostat per day; and about 5 mg of dapagliflozin per day. In some embodiments, the methods comprise administering about 6 mg of verinurad or a pharmaceutically acceptable salt thereof per day; about 80 mg of febuxostat per day; and about 5 mg of dapagliflozin per day. In some embodiments, the methods comprise administering about 7 mg of verinurad or a pharmaceutically acceptable salt thereof per day; about 80 mg of febuxostat per day; and about 5 mg of dapagliflozin per day. In some embodiments, the methods comprise administering about 7.5 mg of verinurad or a pharmaceutically acceptable salt thereof per day; about 80 mg of febuxostat per day; and about 5 mg of dapagliflozin per day. In some embodiments, the methods comprise administering about 8 mg of verinurad or a pharmaceutically acceptable salt thereof per day; about 80 mg of febuxostat per day; and about 5 mg of dapagliflozin per day. In some embodiments, the methods comprise administering about 9 mg of verinurad or a pharmaceutically acceptable salt thereof per day; about 80 mg of febuxostat per day; and about 5 mg of dapagliflozin per day. In some embodiments, the methods comprise administering about 10 mg of verinurad or a pharmaceutically acceptable salt thereof per day; about 80 mg of febuxostat per day; and about 5 mg of dapagliflozin per day. In some embodiments, the methods comprise administering about 11 mg of verinurad or a pharmaceutically acceptable salt thereof per day; about 80 mg of febuxostat per day; and about 5 mg of dapagliflozin per day. In some embodiments, the methods comprise administering about 12 mg of verinurad or a pharmaceutically acceptable salt thereof per day; about 80 mg of febuxostat per day; and about 5 mg of dapagliflozin per day. In some embodiments, the methods comprise administering about 13 mg of verinurad or a pharmaceutically acceptable salt thereof per day; about 80 mg of febuxostat per day; and about 5 mg of dapagliflozin per day. In some embodiments, the methods comprise administering about 14 mg of verinurad or a pharmaceutically acceptable salt thereof per day; about 80 mg of febuxostat per day; and about 5 mg of dapagliflozin per day. In some embodiments, the methods comprise administering about 15 mg of verinurad or a pharmaceutically acceptable salt thereof per day; about 80 mg of febuxostat per day; and about 5 mg of dapagliflozin per day. In some embodiments, the methods comprise administering about 2 mg of verinurad or a pharmaceutically acceptable salt thereof per day; about 80 mg of febuxostat per day; and about 10 mg of dapagliflozin per day. In some embodiments, the methods comprise administering about 3 mg of verinurad or a pharmaceutically acceptable salt thereof per day; about 80 mg of febuxostat per day; and about 10 mg of dapagliflozin per day. In some embodiments, the methods comprise administering about 4 mg of verinurad or a pharmaceutically acceptable salt thereof per day; about 80 mg of febuxostat per day; and about 10 mg of dapagliflozin per day. In some embodiments, the methods comprise administering about 5 mg of verinurad or a pharmaceutically acceptable salt thereof per day; about 80 mg of febuxostat per day; and about 10 mg of dapagliflozin per day. In some embodiments, the methods comprise administering about 6 mg of verinurad or a pharmaceutically acceptable salt thereof per day; about 80 mg of febuxostat per day; and about 10 mg of dapagliflozin per day. In some embodiments, the methods comprise administering about 7 mg of verinurad or a pharmaceutically acceptable salt thereof per day; about 80 mg of febuxostat per day; and about 10 mg of dapagliflozin per day. In some embodiments, the methods comprise administering about 7.5 mg of verinurad or a pharmaceutically acceptable salt thereof per day; about 80 mg of febuxostat; and about 10 mg of dapagliflozin per day. In some embodiments, the methods comprise administering about 8 mg of verinurad or a pharmaceutically acceptable salt thereof per day; about 80 mg of febuxostat per day; and about 10 mg of dapagliflozin per day. In some embodiments, the methods comprise administering about 9 mg of verinurad or a pharmaceutically acceptable salt thereof per day; about 80 mg of febuxostat per day; and about 10 mg of dapagliflozin per day. In some embodiments, the methods comprise administering about 10 mg of verinurad or a pharmaceutically acceptable salt thereof per day; about 80 mg of febuxostat per day; and about 10 mg of dapagliflozin per day. In some embodiments, the methods comprise administering about 11 mg of verinurad or a pharmaceutically acceptable salt thereof per day; about 80 mg of febuxostat per day; and about 10 mg of dapagliflozin per day. In some embodiments, the methods comprise administering about 12 mg of verinurad or a pharmaceutically acceptable salt thereof per day; about 80 mg of febuxostat per day; and about 10 mg of dapagliflozin per day. In some embodiments, the methods comprise administering about 13 mg of verinurad or a pharmaceutically acceptable salt thereof per day; about 80 mg of febuxostat per day; and about 10 mg of dapagliflozin per day. In some embodiments, the methods comprise administering about 14 mg of verinurad or a pharmaceutically acceptable salt thereof per day; about 80 mg of febuxostat per day; and about 10 mg of dapagliflozin per day. In some embodiments, the methods comprise administering about 15 mg of verinurad or a pharmaceutically acceptable salt thereof per day; about 80 mg of febuxostat per day; and about 10 mg of dapagliflozin per day.

In some embodiments, the methods described herein comprise administering a combination of a URAT1 inhibitor (e.g., verinurad or a pharmaceutically acceptable salt thereof) and a xanthine oxidase inhibitor. In some embodiments, the URAT1 inhibitor is verinurad or a pharmaceutically acceptable salt thereof. In some embodiments, the URAT1 inhibitor is verinurad or a pharmaceutically acceptable salt thereof, and the xanthine oxidase inhibitor is febuxostat. In some embodiments, the URAT1 inhibitor is verinurad or a pharmaceutically acceptable salt thereof, and the xanthine oxidase inhibitor is allopurinol.

In some embodiments, the methods comprise administering between 1 mg and 20 mg of verinurad or a pharmaceutically acceptable salt thereof; and between 100 mg and 600 mg of allopurinol. In some embodiments, the methods comprise administering between 5 mg and 15 mg of verinurad or a pharmaceutically acceptable salt thereof; and between 100 mg and 400 mg of allopurinol. In some embodiments, the methods comprise administering about 9 mg of verinurad or a pharmaceutically acceptable salt thereof; and about 100 mg of allopurinol. In some embodiments, the methods comprise administering about 9 mg of verinurad or a pharmaceutically acceptable salt thereof; and about 200 mg of allopurinol. In some embodiments, the methods comprise administering about 9 mg of verinurad or a pharmaceutically acceptable salt thereof; and about 300 mg of allopurinol. In some embodiments, the methods comprise administering about 9 mg of verinurad or a pharmaceutically acceptable salt thereof; and about 400 mg of allopurinol. In some embodiments, the methods comprise administering about 9 mg of verinurad or a pharmaceutically acceptable salt thereof; and about 100 mg of allopurinol. In some embodiments, the methods comprise administering about 9 mg of verinurad or a pharmaceutically acceptable salt thereof; and about 200 mg of allopurinol. In some embodiments, the methods comprise administering about 9 mg of verinurad or a pharmaceutically acceptable salt thereof; and about 300 mg of allopurinol. In some embodiments, the methods comprise administering about 9 mg of verinurad or a pharmaceutically acceptable salt thereof; and about 400 mg of allopurinol. In some embodiments, the methods comprise administering about 9 mg of verinurad or a pharmaceutically acceptable salt thereof; and about 300 mg of allopurinol. In some embodiments, the methods comprise administering about 2 mg of verinurad or a pharmaceutically acceptable salt thereof; and about 300 mg of allopurinol. In some embodiments, the methods comprise administering about 3 mg of verinurad or a pharmaceutically acceptable salt thereof; and about 300 mg of allopurinol. In some embodiments, the methods comprise administering about 4 mg of verinurad or a pharmaceutically acceptable salt thereof; and about 300 mg of allopurinol. In some embodiments, the methods comprise administering about 5 mg of verinurad or a pharmaceutically acceptable salt thereof; and about 300 mg of allopurinol. In some embodiments, the methods comprise administering about 6 mg of verinurad or a pharmaceutically acceptable salt thereof; and about 300 mg of allopurinol. In some embodiments, the methods comprise administering about 7 mg of verinurad or a pharmaceutically acceptable salt thereof; and about 300 mg of allopurinol. In some embodiments, the methods comprise administering about 7.5 mg of verinurad or a pharmaceutically acceptable salt thereof; and about 300 mg of allopurinol. In some embodiments, the methods comprise administering about 8 mg of verinurad or a pharmaceutically acceptable salt thereof; and about 300 mg of allopurinol. In some embodiments, the methods comprise administering about 9 mg of verinurad or a pharmaceutically acceptable salt thereof; and about 300 mg of allopurinol. In some embodiments, the methods comprise administering about 10 mg of verinurad or a pharmaceutically acceptable salt thereof; and about 300 mg of allopurinol. In some embodiments, the methods comprise administering about 11 mg of verinurad or a pharmaceutically acceptable salt thereof; and about 300 mg of allopurinol. In some embodiments, the methods comprise administering about 12 mg of verinurad or a pharmaceutically acceptable salt thereof; and about 300 mg of allopurinol. In some embodiments, the methods comprise administering about 13 mg of verinurad or a pharmaceutically acceptable salt thereof; and about 300 mg of allopurinol. In some embodiments, the methods comprise administering about 14 mg of verinurad or a pharmaceutically acceptable salt thereof; and about 300 mg of allopurinol. In some embodiments, the methods comprise administering about 15 mg of verinurad or a pharmaceutically acceptable salt thereof; and about 300 mg of allopurinol. In some embodiments, the methods comprise administering about 2 mg of verinurad or a pharmaceutically acceptable salt thereof; and about 300 mg of allopurinol. In some embodiments, the methods comprise administering about 3 mg of verinurad or a pharmaceutically acceptable salt thereof; and about 300 mg of allopurinol. In some embodiments, the methods comprise administering about 4 mg of verinurad or a pharmaceutically acceptable salt thereof; and about 300 mg of allopurinol. In some embodiments, the methods comprise administering about 5 mg of verinurad or a pharmaceutically acceptable salt thereof; and about 300 mg of allopurinol. In some embodiments, the methods comprise administering about 6 mg of verinurad or a pharmaceutically acceptable salt thereof; and about 300 mg of allopurinol. In some embodiments, the methods comprise administering about 7 mg of verinurad or a pharmaceutically acceptable salt thereof; and about 300 mg of allopurinol. In some embodiments, the methods comprise administering about 7.5 mg of verinurad or a pharmaceutically acceptable salt thereof; and about 300 mg of allopurinol. In some embodiments, the methods comprise administering about 8 mg of verinurad or a pharmaceutically acceptable salt thereof; and about 300 mg of allopurinol. In some embodiments, the methods comprise administering about 9 mg of verinurad or a pharmaceutically acceptable salt thereof; and about 300 mg of allopurinol. In some embodiments, the methods comprise administering about 10 mg of verinurad or a pharmaceutically acceptable salt thereof; and about 300 mg of allopurinol. In some embodiments, the methods comprise administering about 11 mg of verinurad or a pharmaceutically acceptable salt thereof; and about 300 mg of allopurinol. In some embodiments, the methods comprise administering about 12 mg of verinurad or a pharmaceutically acceptable salt thereof; and about 300 mg of allopurinol. In some embodiments, the methods comprise administering about 13 mg of verinurad or a pharmaceutically acceptable salt thereof; and about 300 mg of allopurinol. In some embodiments, the methods comprise administering about 14 mg of verinurad or a pharmaceutically acceptable salt thereof; and about 300 mg of allopurinol. In some embodiments, the methods comprise administering about 15 mg of verinurad or a pharmaceutically acceptable salt thereof; and about 300 mg of allopurinol.

In some embodiments, the methods comprise administering between 1 mg and 20 mg of verinurad or a pharmaceutically acceptable salt thereof per day; and between 100 mg and 600 mg of allopurinol per day. In some embodiments, the methods comprise administering between 5 mg and 15 mg of verinurad or a pharmaceutically acceptable salt thereof per day; and between 100 mg and 400 mg of allopurinol per day. In some embodiments, the methods comprise administering about 9 mg of verinurad or a pharmaceutically acceptable salt thereof per day; and about 100 mg of allopurinol per day. In some embodiments, the methods comprise administering about 9 mg of verinurad or a pharmaceutically acceptable salt thereof per day; and about 200 mg of allopurinol per day. In some embodiments, the methods comprise administering about 9 mg of verinurad or a pharmaceutically acceptable salt thereof per day; and about 300 mg of allopurinol per day. In some embodiments, the methods comprise administering about 9 mg of verinurad or a pharmaceutically acceptable salt thereof per day; and about 400 mg of allopurinol per day. In some embodiments, the methods comprise administering about 9 mg of verinurad or a pharmaceutically acceptable salt thereof per day; and about 100 mg of allopurinol per day. In some embodiments, the methods comprise administering about 9 mg of verinurad or a pharmaceutically acceptable salt thereof per day; and about 200 mg of allopurinol per day. In some embodiments, the methods comprise administering about 9 mg of verinurad or a pharmaceutically acceptable salt thereof per day; and about 300 mg of allopurinol per day. In some embodiments, the methods comprise administering about 9 mg of verinurad or a pharmaceutically acceptable salt thereof per day; and about 400 mg of allopurinol per day. In some embodiments, the methods comprise administering about 9 mg of verinurad or a pharmaceutically acceptable salt thereof per day; and about 300 mg of allopurinol per day. In some embodiments, the methods comprise administering about 2 mg of verinurad or a pharmaceutically acceptable salt thereof per day; and about 300 mg of allopurinol per day. In some embodiments, the methods comprise administering about 3 mg of verinurad or a pharmaceutically acceptable salt thereof per day; and about 300 mg of allopurinol per day. In some embodiments, the methods comprise administering about 4 mg of verinurad or a pharmaceutically acceptable salt thereof per day; and about 300 mg of allopurinol per day. In some embodiments, the methods comprise administering about 5 mg of verinurad or a pharmaceutically acceptable salt thereof per day; and about 300 mg of allopurinol per day. In some embodiments, the methods comprise administering about 6 mg of verinurad or a pharmaceutically acceptable salt thereof per day; and about 300 mg of allopurinol per day. In some embodiments, the methods comprise administering about 7 mg of verinurad or a pharmaceutically acceptable salt thereof per day; and about 300 mg of allopurinol per day. In some embodiments, the methods comprise administering about 7.5 mg of verinurad or a pharmaceutically acceptable salt thereof per day; and about 300 mg of allopurinol per day. In some embodiments, the methods comprise administering about 8 mg of verinurad or a pharmaceutically acceptable salt thereof per day; and about 300 mg of allopurinol per day. In some embodiments, the methods comprise administering about 9 mg of verinurad or a pharmaceutically acceptable salt thereof per day; and about 300 mg of allopurinol per day. In some embodiments, the methods comprise administering about 10 mg of verinurad or a pharmaceutically acceptable salt thereof per day; and about 300 mg of allopurinol per day. In some embodiments, the methods comprise administering about 11 mg of verinurad or a pharmaceutically acceptable salt thereof per day; and about 300 mg of allopurinol per day. In some embodiments, the methods comprise administering about 12 mg of verinurad or a pharmaceutically acceptable salt thereof per day; and about 300 mg of allopurinol per day. In some embodiments, the methods comprise administering about 13 mg of verinurad or a pharmaceutically acceptable salt thereof per day; and about 300 mg of allopurinol per day. In some embodiments, the methods comprise administering about 14 mg of verinurad or a pharmaceutically acceptable salt thereof per day; and about 300 mg of allopurinol per day. In some embodiments, the methods comprise administering about 15 mg of verinurad or a pharmaceutically acceptable salt thereof per day; and about 300 mg of allopurinol per day. In some embodiments, the methods comprise administering about 2 mg of verinurad or a pharmaceutically acceptable salt thereof per day; and about 300 mg of allopurinol per day. In some embodiments, the methods comprise administering about 3 mg of verinurad or a pharmaceutically acceptable salt thereof per day; and about 300 mg of allopurinol per day. In some embodiments, the methods comprise administering about 4 mg of verinurad or a pharmaceutically acceptable salt thereof per day; and about 300 mg of allopurinol per day. In some embodiments, the methods comprise administering about 5 mg of verinurad or a pharmaceutically acceptable salt thereof per day; and about 300 mg of allopurinol per day. In some embodiments, the methods comprise administering about 6 mg of verinurad or a pharmaceutically acceptable salt thereof per day; and about 300 mg of allopurinol per day. In some embodiments, the methods comprise administering about 7 mg of verinurad or a pharmaceutically acceptable salt thereof per day; and about 300 mg of allopurinol per day. In some embodiments, the methods comprise administering about 7.5 mg of verinurad or a pharmaceutically acceptable salt thereof per day; and about 300 mg of allopurinol per day. In some embodiments, the methods comprise administering about 8 mg of verinurad or a pharmaceutically acceptable salt thereof per day; and about 300 mg of allopurinol per day. In some embodiments, the methods comprise administering about 9 mg of verinurad or a pharmaceutically acceptable salt thereof per day; and about 300 mg of allopurinol per day. In some embodiments, the methods comprise administering about 10 mg of verinurad or a pharmaceutically acceptable salt thereof per day; and about 300 mg of allopurinol per day. In some embodiments, the methods comprise administering about 11 mg of verinurad or a pharmaceutically acceptable salt thereof per day; and about 300 mg of allopurinol per day. In some embodiments, the methods comprise administering about 12 mg of verinurad or a pharmaceutically acceptable salt thereof per day; and about 300 mg of allopurinol per day. In some embodiments, the methods comprise administering about 13 mg of verinurad or a pharmaceutically acceptable salt thereof per day; and about 300 mg of allopurinol per day. In some embodiments, the methods comprise administering about 14 mg of verinurad or a pharmaceutically acceptable salt thereof per day; and about 300 mg of allopurinol per day. In some embodiments, the methods comprise administering about 15 mg of verinurad or a pharmaceutically acceptable salt thereof per day; and about 300 mg of allopurinol per day.

In some embodiments, the methods described herein comprise administering between 1 mg and 20 mg of verinurad or a pharmaceutically acceptable salt thereof; and between 10 mg and 200 mg of febuxostat. In some embodiments, the methods comprise administering between 5 mg and 15 mg of verinurad or a pharmaceutically acceptable salt thereof; and between 40 mg and 100 mg of febuxostat. In some embodiments, the methods comprise administering about 9 mg of verinurad or a pharmaceutically acceptable salt thereof; and about 40 mg of febuxostat. In some embodiments, the methods comprise administering about 9 mg of verinurad or a pharmaceutically acceptable salt thereof; and about 80 mg of febuxostat. In some embodiments, the methods comprise administering about 9 mg of verinurad or a pharmaceutically acceptable salt thereof; and about 40 mg of febuxostat. In some embodiments, the methods comprise administering about 9 mg of verinurad or a pharmaceutically acceptable salt thereof; and about 80 mg of febuxostat. In some embodiments, the methods comprise administering about 2 mg of verinurad or a pharmaceutically acceptable salt thereof; and about 80 mg of febuxostat. In some embodiments, the methods comprise administering about 3 mg of verinurad or a pharmaceutically acceptable salt thereof; and about 80 mg of febuxostat. In some embodiments, the methods comprise administering about 4 mg of verinurad or a pharmaceutically acceptable salt thereof; and about 80 mg of febuxostat. In some embodiments, the methods comprise administering about 5 mg of verinurad or a pharmaceutically acceptable salt thereof; and about 80 mg of febuxostat. In some embodiments, the methods comprise administering about 6 mg of verinurad or a pharmaceutically acceptable salt thereof; and about 80 mg of febuxostat. In some embodiments, the methods comprise administering about 7 mg of verinurad or a pharmaceutically acceptable salt thereof; and about 80 mg of febuxostat. In some embodiments, the methods comprise administering about 7.5 mg of verinurad or a pharmaceutically acceptable salt thereof; and about 80 mg of febuxostat. In some embodiments, the methods comprise administering about 8 mg of verinurad or a pharmaceutically acceptable salt thereof; and about 80 mg of febuxostat. In some embodiments, the methods comprise administering about 9 mg of verinurad or a pharmaceutically acceptable salt thereof; and about 80 mg of febuxostat. In some embodiments, the methods comprise administering about 10 mg of verinurad or a pharmaceutically acceptable salt thereof; and about 80 mg of febuxostat. In some embodiments, the methods comprise administering about 11 mg of verinurad or a pharmaceutically acceptable salt thereof; and about 80 mg of febuxostat. In some embodiments, the methods comprise administering about 12 mg of verinurad or a pharmaceutically acceptable salt thereof; and about 80 mg of febuxostat. In some embodiments, the methods comprise administering about 13 mg of verinurad or a pharmaceutically acceptable salt thereof; and about 80 mg of febuxostat. In some embodiments, the methods comprise administering about 14 mg of verinurad or a pharmaceutically acceptable salt thereof; and about 80 mg of febuxostat. In some embodiments, the methods comprise administering about 15 mg of verinurad or a pharmaceutically acceptable salt thereof; and about 80 mg of febuxostat. In some embodiments, the methods comprise administering about 2 mg of verinurad or a pharmaceutically acceptable salt thereof; and about 80 mg of febuxostat. In some embodiments, the methods comprise administering about 3 mg of verinurad or a pharmaceutically acceptable salt thereof; and about 80 mg of febuxostat. In some embodiments, the methods comprise administering about 4 mg of verinurad or a pharmaceutically acceptable salt thereof; and about 80 mg of febuxostat. In some embodiments, the methods comprise administering about 5 mg of verinurad or a pharmaceutically acceptable salt thereof; and about 80 mg of febuxostat. In some embodiments, the methods comprise administering about 6 mg of verinurad or a pharmaceutically acceptable salt thereof; and about 80 mg of febuxostat. In some embodiments, the methods comprise administering about 7 mg of verinurad or a pharmaceutically acceptable salt thereof; and about 80 mg of febuxostat. In some embodiments, the methods comprise administering about 7.5 mg of verinurad or a pharmaceutically acceptable salt thereof; and about 80 mg of febuxostat. In some embodiments, the methods comprise administering about 8 mg of verinurad or a pharmaceutically acceptable salt thereof; and about 80 mg of febuxostat. In some embodiments, the methods comprise administering about 9 mg of verinurad or a pharmaceutically acceptable salt thereof; and about 80 mg of febuxostat. In some embodiments, the methods comprise administering about 10 mg of verinurad or a pharmaceutically acceptable salt thereof; and about 80 mg of febuxostat. In some embodiments, the methods comprise administering about 11 mg of verinurad or a pharmaceutically acceptable salt thereof; and about 80 mg of febuxostat. In some embodiments, the methods comprise administering about 12 mg of verinurad or a pharmaceutically acceptable salt thereof; and about 80 mg of febuxostat. In some embodiments, the methods comprise administering about 13 mg of verinurad or a pharmaceutically acceptable salt thereof; and about 80 mg of febuxostat. In some embodiments, the methods comprise administering about 14 mg of verinurad or a pharmaceutically acceptable salt thereof; and about 80 mg of febuxostat. In some embodiments, the methods comprise administering about 15 mg of verinurad or a pharmaceutically acceptable salt thereof; and about 80 mg of febuxostat.

In some embodiments, the methods described herein comprise administering between 1 mg and 20 mg of verinurad or a pharmaceutically acceptable salt thereof per day; and between 10 mg and 200 mg of febuxostat per day. In some embodiments, the methods comprise administering between 5 mg and 15 mg of verinurad or a pharmaceutically acceptable salt thereof per day; and between 40 mg and 100 mg of febuxostat per day. In some embodiments, the methods comprise administering about 9 mg of verinurad or a pharmaceutically acceptable salt thereof per day; and about 40 mg of febuxostat per day. In some embodiments, the methods comprise administering about 9 mg of verinurad or a pharmaceutically acceptable salt thereof per day; and about 80 mg of febuxostat per day. In some embodiments, the methods comprise administering about 9 mg of verinurad or a pharmaceutically acceptable salt thereof per day; and about 40 mg of febuxostat per day. In some embodiments, the methods comprise administering about 9 mg of verinurad or a pharmaceutically acceptable salt thereof per day; and about 80 mg of febuxostat per day. In some embodiments, the methods comprise administering about 2 mg of verinurad or a pharmaceutically acceptable salt thereof per day; and about 80 mg of febuxostat per day. In some embodiments, the methods comprise administering about 3 mg of verinurad or a pharmaceutically acceptable salt thereof per day; and about 80 mg of febuxostat per day. In some embodiments, the methods comprise administering about 4 mg of verinurad or a pharmaceutically acceptable salt thereof per day; and about 80 mg of febuxostat per day. In some embodiments, the methods comprise administering about 5 mg of verinurad or a pharmaceutically acceptable salt thereof per day; and about 80 mg of febuxostat per day. In some embodiments, the methods comprise administering about 6 mg of verinurad or a pharmaceutically acceptable salt thereof per day; and about 80 mg of febuxostat per day. In some embodiments, the methods comprise administering about 7 mg of verinurad or a pharmaceutically acceptable salt thereof per day; and about 80 mg of febuxostat per day. In some embodiments, the methods comprise administering about 7.5 mg of verinurad or a pharmaceutically acceptable salt thereof per day; and about 80 mg of febuxostat per day. In some embodiments, the methods comprise administering about 8 mg of verinurad or a pharmaceutically acceptable salt thereof per day; and about 80 mg of febuxostat per day. In some embodiments, the methods comprise administering about 9 mg of verinurad or a pharmaceutically acceptable salt thereof per day; and about 80 mg of febuxostat per day. In some embodiments, the methods comprise administering about 10 mg of verinurad or a pharmaceutically acceptable salt thereof per day; and about 80 mg of febuxostat per day. In some embodiments, the methods comprise administering about 11 mg of verinurad or a pharmaceutically acceptable salt thereof per day; and about 80 mg of febuxostat per day. In some embodiments, the methods comprise administering about 12 mg of verinurad or a pharmaceutically acceptable salt thereof per day; and about 80 mg of febuxostat per day. In some embodiments, the methods comprise administering about 13 mg of verinurad or a pharmaceutically acceptable salt thereof per day; and about 80 mg of febuxostat per day. In some embodiments, the methods comprise administering about 14 mg of verinurad or a pharmaceutically acceptable salt thereof per day; and about 80 mg of febuxostat per day. In some embodiments, the methods comprise administering about 15 mg of verinurad or a pharmaceutically acceptable salt thereof per day; and about 80 mg of febuxostat per day. In some embodiments, the methods comprise administering about 2 mg of verinurad or a pharmaceutically acceptable salt thereof per day; and about 80 mg of febuxostat per day. In some embodiments, the methods comprise administering about 3 mg of verinurad or a pharmaceutically acceptable salt thereof per day; and about 80 mg of febuxostat per day. In some embodiments, the methods comprise administering about 4 mg of verinurad or a pharmaceutically acceptable salt thereof per day; and about 80 mg of febuxostat per day. In some embodiments, the methods comprise administering about 5 mg of verinurad or a pharmaceutically acceptable salt thereof per day; and about 80 mg of febuxostat per day. In some embodiments, the methods comprise administering about 6 mg of verinurad or a pharmaceutically acceptable salt thereof per day; and about 80 mg of febuxostat per day. In some embodiments, the methods comprise administering about 7 mg of verinurad or a pharmaceutically acceptable salt thereof per day; and about 80 mg of febuxostat per day. In some embodiments, the methods comprise administering about 7.5 mg of verinurad or a pharmaceutically acceptable salt thereof per day; and about 80 mg of febuxostat. In some embodiments, the methods comprise administering about 8 mg of verinurad or a pharmaceutically acceptable salt thereof per day; and about 80 mg of febuxostat per day. In some embodiments, the methods comprise administering about 9 mg of verinurad or a pharmaceutically acceptable salt thereof per day; and about 80 mg of febuxostat per day. In some embodiments, the methods comprise administering about 10 mg of verinurad or a pharmaceutically acceptable salt thereof per day; and about 80 mg of febuxostat per day. In some embodiments, the methods comprise administering about 11 mg of verinurad or a pharmaceutically acceptable salt thereof per day; and about 80 mg of febuxostat per day. In some embodiments, the methods comprise administering about 12 mg of verinurad or a pharmaceutically acceptable salt thereof per day; and about 80 mg of febuxostat per day. In some embodiments, the methods comprise administering about 13 mg of verinurad or a pharmaceutically acceptable salt thereof per day; and about 80 mg of febuxostat per day. In some embodiments, the methods comprise administering about 14 mg of verinurad or a pharmaceutically acceptable salt thereof per day; and about 80 mg of febuxostat per day. In some embodiments, the methods comprise administering about 15 mg of verinurad or a pharmaceutically acceptable salt thereof per day; and about 80 mg of febuxostat per day.

In some embodiments, because of the combination therapy, it may be possible to administer lesser amounts of a component (for example, the URAT1 inhibitor, XOI, and/or SGLT2 inhibitor) and still have therapeutic or prophylactic effect.

### Pharmaceutical Compositions

The instant disclosure provides pharmaceutical compositions comprising active ingredients described herein for use in the methods described herein. In some embodiments, the pharmaceutical compositions comprise an effective amount of a URAT1 inhibitor, an XOI, and optionally an SGLT2 inhibitor. In some embodiments, the pharmaceutical compositions comprise an effective amount of a URAT1 inhibitor, an XOI, and optionally an SGLT2 inhibitor and at least one pharmaceutically acceptable carrier or excipient. In some embodiments, the pharmaceutical compositions are useful for treating or preventing a condition disclosed herein. In some embodiments, the pharmaceutical compositions are for the treatment of disorders in a human.

### Formulations

The pharmaceutical compositions described herein can also contain the active ingredients in a form suitable for oral use, for example, as tablets, troches, lozenges, aqueous or oily suspensions, dispersible powders or granules, emulsions, hard or soft capsules, or syrups or elixirs. Compositions intended for oral use are optionally prepared according to known method, and such compositions may contain one or more agents selected from the group consisting of sweetening agents, flavoring agents, coloring agents and preserving agents in order to provide pharmaceutically elegant and palatable preparations.

Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be un-coated or coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate may be employed as appropriate.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-5 oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening agents, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

### Dosage Forms

For convenience, the total daily dosage may be divided and administered in portions during the day if desired. Accordingly, the total daily dose may be subdivided into unit doses containing appropriate quantities of the active components, e.g. an effective amount of a URAT1 inhibitor, XOI, and optionally a SGLT2 inhibitor (“active ingredients”) to achieve the desired purpose. Unit dosage forms may be prepared by any of the methods well known in the art of pharmacy. In general, unit dosage forms may be prepared similarly to the formulations described herein.

Dosage forms may, for example, be in a form suitable for oral administration as a tablet, capsule, pill, powder, sustained release formulations, solution, or suspension. Dosage forms may include a pharmaceutically acceptable carrier or excipient and a URAT1 inhibitor, XOI, and optionally an SGLT2 inhibitor as described herein as an active ingredient. In addition, they may include other medicinal or pharmaceutical agents, carriers, adjuvants, etc. In some embodiments, the active ingredients are administered in separate dosage units. In some embodiments, for example, each active ingredient is administered in a separate tablet. In other embodiments, the active ingredients are administered in a single dosage unit. For example, all active ingredients may be administered in the same tablet. In some embodiments, the unit dosage form comprises, for example, three separate tablets individually comprising one active ingredient), two tablets wherein one tablet comprises two of active ingredients and the other tablet comprising a third active ingredient), or a single tablet which comprises all active ingredients.

### Kits

The instant disclosure further provides kits for use in the methods described herein. These kits comprise compounds or compositions described herein in a container and, optionally, instructions teaching the use of the kit according to the various methods and approaches described herein. In some embodiments, such kits also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. In some embodiments, such information is based on the results of various studies, for example, studies using experimental animals involving in vivo models and studies based on human clinical trials. In some embodiments, kits described herein are provided, marketed and/or promoted to health providers, including physicians, nurses, pharmacists, formulary officials, and the like. In some embodiments, kits are marketed directly to the consumer.

### EXAMPLES

### Example 1

### A Study to Assess the Effect of Intensive Uric Acid (UA) Lowering Therapy with Verinurad, Febuxostat, Dapagliflozin on Urinary Excretion of UA

This study was a randomized, placebo controlled, double-blind, 2-way crossover study conducted on asymptomatic hyperuricemic patients. The core study consists of screening period, two treatment periods (verinurad+febuxostat+dapagliflozin/placebo), and follow-up visit.

| Condition | Intervention | Phase |
|---|---|---|
| Asymptomatic | Drug: Verinurad | Phase 2 |
| Hyperuricemia | Drug: Febuxostat | |
| | Drug: Dapagliflozin | |
| | Other: Dapagliflozin | |
| | matched placebo | |

Study Type: Interventional (Clinical Trial)
Actual Enrollment: 36 participants
Allocation: Randomized
Intervention Model: Crossover Assignment
Masking: Double (Participant, Outcomes Assessor) [The pharmacokineticist will remain blinded during the study conduct, unless otherwise required based on study findings. The pharmacokineticist will be unblinded to perform the final PK analyses after all patients have completed the study, final bioanalytical results are available and all required study data are considered clean. This may occur before database lock.]
Primary Purpose: Treatment

### DETAILED DESCRIPTION

This study was a randomized, placebo controlled, double-blind, 2-way crossover study to assess the effect of intensive UA lowering therapy with verinurad, febuxostat, and dapagliflozin on urinary excretion of UA, in asymptomatic hyperuricemic patients. Thirty-six asymptomatic hyperuricemic patients aged 18 to 65 years (inclusive) were enrolled into this study at two study centers. Twenty-four patients were enrolled and completed the study. Due to inadequate urine sampling, 12 additional patients were included to ensure an adequate sample size (at least 20 evaluable patients) to evaluate the effects of intensive UA lowering with verinurad, febuxostat and dapagliflozin on urinary excretion of UA. With 24 completers available during the interim analysis, this will provide for a total sample size of 36 evaluable patients.

Before any study specific assessments were performed, potential patients provided informed consent. Each patient underwent the below mentioned visits:
A Screening period of maximum 28 days;
Two treatment periods during which patients were resident in the Clinical Unit from Day −2 to Day 1 and from Day 6 to Day 8; and
A Follow-up Visit within 14 to 28 days after the first administration of Investigational Medicinal Product (IMP) in Treatment Period 2.

On Day −2 of Treatment period 1, patients were randomized (1:1) to 1 of 2 treatment sequences (AB or BA). Each randomized patient received orally once daily fixed dose of the below mentioned 2 treatments for 7 consecutive days (1 treatment per treatment period).
Treatment A: verinurad+febuxostat+dapagliflozin
Treatment B: verinurad+febuxostat+placebo
For each treatment period, baseline measurements were performed. On Day 1, after all dosing and all assessments were performed, patients received instruction to administer the IMP at home once daily in the morning from Day 2 to Day 6 and the IMP dispensed for home dosing. Patients returned to the Clinical Unit on Day 6 and were residential in the Clinical Unit from Day 6 to Day 8.

Treatment Period 1 and Treatment Period 2 were separated by a washout period of 7 to 21 days.

Patients returned to the Clinical Unit for a Follow-up Visit, 14 to 28 days after Day 1 of Treatment Period 2.

### Arms and Interventions

| Arms | Assigned Interventions |
|---|---|
| Experimental: Treatment A | Drug: Verinurad |
| Randomized patients received | Dose: 9 mg/day |
| orally once daily fixed dose of | Drug: Febuxostat |
| the following drugs: | Other Name: ULORIC ® |
| verinurad + febuxostat + | Dose: 80 mg/day |
| dapagliflozin | Drug: Dapagliflozin |
| | Other Name: FARXIGA |
| | Dose: 10 mg/day |
| | Randomized patients received orally |
| | once daily fixed dose of verinurad, |
| | febuxostat, and dapagliflozin in 2 |
| | treatment sequences AB or BA for 7 |
| | consecutive days. |
| | Treatment A: verinurad + febuxostat + |
| | dapagliflozin; |
| | Treatment B: verinurad + febuxostat + |
| | placebo |
| Experimental: Treatment B | Drug: Verinurad |
| Randomized patients received | Dose: 9 mg/day |
| orally once daily fixed dose of | Drug: Febuxostat |
| the following drugs: | Other Name: ULORIC ® |
| verinurad + febuxostat + | Dose: 80 mg/day |
| dapagliflozin matched placebo | Drug: Dapagliflozin matched placebo |
| | Randomized patients received orally |
| | once daily fixed dose of verinurad, |
| | febuxostat, and dapagliflozin matched |
| | placebo in 2 treatment sequences AB or |
| | BA for 7 consecutive days. |
| | Treatment A: verinurad + febuxostat + |
| | dapagliflozin; |
| | Treatment B: verinurad + febuxostat + |
| | placebo |

### Primary Outcome Measures

1. Effect of verinurad, febuxostat, and dapagliflozin on urinary excretion of uric acid (UA) on Day 7 [Time Frame: On Treatment Period 1 and 2: Day −1 and Day 7].
   To assess the difference in within-subject change from baseline in peak UA excretion during the first 8 hours (maximum UA excreted as measured in milligram [mg], in an interval out of the first 8 hours) on Day 7 of treatment (Day 1 is the first day of treatment).
   On Day −1: Hourly baseline collection of urine from −24 to −12 hours followed by a single 12-hour collection from −12 to 0 hours (0 hours is time of dosing on Day 1) On Day 7: Directly after the dose of study treatment, hourly collection of urine is performed every hour from 0 to 12 hours (inclusive) followed by a single pooled collection from 12 to 24 hours.

### Secondary Outcome Measures

1. Effects of verinurad, febuxostat, and dapagliflozin on urinary excretion of serum uric acid (sUA) after 7 days of treatment [Time Frame: At screening and Treatment Period 1 and 2: Day −1 and Day 7].
   To assess the intensive UA lowering effect of verinurad, febuxostat, and dapagliflozin by evaluating the sUA levels after 7 days of treatment. At screening: One sUA assessment. At treatment period: Always in the morning, and after a 10 hour overnight fast.
2. Area under plasma concentration time curve over a dosing interval (24 hours) (AUC_{τ}) assessment for verinurad and its main metabolites, febuxostat, and dapagliflozin [Time Frame: On Treatment Period 1 and 2: Day 7 (Pre-dose and 15 minutes, 30 minutes, 1 hour, 1.5, 2, 3, 4, 8, 12 and 24 hours post-dose)]
   To assess AUCT after administration of single oral fixed dose of verinurad+febuxostat+dapagliflozin/placebo
3. Maximum observed plasma concentration (Cₘₐₓ) assessment for verinurad and its main metabolites, febuxostat, and dapagliflozin [Time Frame: On Treatment Period 1 and 2: Day 7 (Pre-dose and 15 minutes, 30 minutes, 1 hour, 1.5, 2, 3, 4, 8, 12 and 24 hours post-dose)]
   To assess Cₘₐₓ after administration of single oral dose of verinurad+febuxostat+dapagliflozin/placebo
4. Number of participants with adverse events (AEs) due to verinurad, febuxostat, and dapagliflozin [Time Frame: At screening (Day −28), Treatment Period 1 (Day -2), Treatment Periods 1 & 2 (Days −1, 1, 6, 7 and 8) and follow-up visit/early discontinuation visit (EDV) (Day 23)]
   To assess the renal and general safety and tolerability of intensive UA lowering therapy with verinurad, febuxostat, and dapagliflozin. AEs will be collected from the start of screening throughout the treatment period up to and including the Follow-up Visit. Serious adverse events will be recorded from the time of informed consent.
5. Systolic blood pressure [SBP] [Time Frame: At screening (Day −28), Treatment Period 1 & 2 (Days −1 and 7) and follow-up visit/EDV (Day 23)]
   To assess SBP as a measure of safety and tolerability.
6. Diastolic blood pressure [DBP] [Time Frame: At screening (Day −28), Treatment Period 1 & 2 (Days −1 and 7) and follow-up visit/EDV (Day 23)]
   To assess DBP as a measure of safety and tolerability.
7. Pulse rate [Time Frame: At screening (Day −28), Treatment Period 1 & 2 (Days −1 and 7) and follow-up visit/EDV (Day 23)]
   To assess pulse as a measure of safety and tolerability.
8. Laboratory assessments of Hematology [Time Frame: At screening (day −28), Treatment Period 1 (Days −2, −1 (only FPG) & 7), Treatment Period 2 (Days −1, 1 (only FPG) & 7) and follow-up visit/EDV (Day 23)]
   To assess the hematology—blood cells count [white blood cell (WBC), red blood cell (RBC), hematocrit (HCT), mean corpuscular volume (MCV), mean corpuscular hemoglobin (MCH) and mean corpuscular hemoglobin concentration (MCHC)], differential absolute count (neutrophils, lymphocytes, eosinophils, basophils, platelets and reticulocytes), hemoglobin (Hb) count and hemoglobin A1c (HbA1c, at screening Visit only) as a measure of safety and tolerability variables. Fasting samples, collected before breakfast (food) and before dose (when applicable).
9. Laboratory assessments of Clinical chemistry [Time Frame: At screening (day −28), Treatment Period 1 (Days −2, −1 (only FPG) & 7), Treatment Period 2 (Days −1, 1 (only FPG) & 7) and follow-up visit/EDV (Day 23)]
   To assess the clinical chemistry (sodium, potassium, blood urea nitrogen (BUN), creatinine, albumin, calcium, phosphate, UA, liver enzymes, total and unconjugated bilirubin; cystatin-C and estimated glomerular filtration rate (eGFR) (glucose—fasting, done at screening only) as a measure of safety and tolerability.
10. Laboratory assessments of urinalysis [Time Frame: At screening (Day −28) and follow-up/EDV (Day 23)]
   To assess the urinalysis (glucose, protein, blood, UA, sodium, pH, creatinine and cystatin-C) as a measure of safety and tolerability variables. If urinalysis is positive for protein or blood, a microscopy test will be performed to assess RBC, WBC, casts [cellular, granular, hyaline]).

### Eligibility

Ages Eligible for Study: 18 Years to 99 Years (Adult, Older Adult)
Sexes Eligible for Study: All
Accepts Healthy Volunteers: No

### Criteria

### Inclusion Criteria:

18 to 65 years old
Asymptomatic hyperuricemia (sUA >6.0 mg/dL)
Body mass index between 18 and 35 kg/m2 inclusive and weight at least 50 kg and no more than 150 kg
Females must be non-pregnant, as well as post-menopausal or willing to use an acceptable method of contraception during the study.

### Exclusion Criteria:

History of any clinically significant disease or disorder putting the patient at risk during the study, or influencing study results or ability to participate in the study
eGFR* <45 mL/minute/1.73 m2 at Screening
Type 2 diabetes mellitus with HbA1c >8%
History of diabetic ketoacidosis, hyperosmolar non-ketotic coma, gout, or alcohol or drug abuse
Ongoing treatment with an SGLT2 inhibitor, a URAT1 inhibitor, and/or a xanthine oxidase inhibitor
Positive test for hepatitis B, hepatitis C or HIV
Use of any medications in the 2 weeks preceding first administration of study drug

### Results

Enrolled subjects (n=24) were male, mean age 43y. At baseline (BL) (D-2), median body mass index (BMI) and eGFR were 29 kg/m² and 85 mL/min/1.73m² with mean (SD) sUA 445.3 (60.8) μmol/L. BL levels of serum creatinine (sCr) and serum sodium (sNa) were (Grp1/Grp2) 93.8/95.8 μmol/L and 138/139 mmol/L, respectively. Peak urinary uric acid (uUA) was available for 13 subjects and 24 h urine for all. After dapagliflozin treatment, subjects showed no increase in peak uUA excretion on D7 (mg/h); Grp1 −5.33 (−15.04, 4.37), Grp2 −7.20 (−16.91, 2.51); mean difference 1.87 (−7.63, 11.36). Similarly, no difference was seen in total 24 h uUA excretion. After dapagliflozin treatment, subjects showed significantly reduced sUA levels; mean difference (Grp1 vs Grp2) on D7 was −76.35 (−104.02, −48.68) μmol/L (p<0.01). No between-group differences were seen in sCr or sNa. 8 subjects had 9 AE (Grp1 n=5; Grp2, n=4), all mild, no inter-group differences. Dapagliflozin did not affect verinurad or febuxostat pharmacokinetics.

### Conclusions

Excessive urinary uric acid excretion may damage renal tubules due to crystallization of uric acid at high concentrations. The effects of administering dapagliflozin in combination with verinurad and febuxostat on sUA and urinary UA (uUA) excretion in subjects with hyperuricemia were examined. Addition of dapagliflozin to an intensive UA lowering strategy with verinurad and febuxostat provides additional sUA lowering without an increase in uUA excretion, or changes in sCr, suggesting that combination therapy with dapagliflozin, verinurad, and febuxostat will not adversely affect kidney function.

While certain embodiments of the claims have been shown and described herein, it will be obvious to those skilled in the art that such embodiments are provided by way of example only. Numerous variations, changes, and substitutions will occur to those skilled in the art without departing from the invention. It should be understood that various alternatives to the embodiments described herein are, in some circumstances, employed in practicing the invention.

It is intended that methods and structures within the scope of these claims and their equivalents be covered thereby. The section headings used herein are for organizational purposes only and are not to be construed as limiting the subject matter described and claimed. All documents, or portions of documents, cited in the application including, without limitation, patents, patent applications, articles, books, manuals, and treatises are hereby expressly incorporated by reference in their entirety for any purpose.

What is claimed is:

1. A method of reducing serum uric acid levels in a subject in need thereof, comprising administering to the subject:
verinurad or a pharmaceutically acceptable salt thereof;
a xanthine oxidase inhibitor; and
dapagliflozin.

2. A method of treating or preventing a condition associated with hyperuricemia in a subject in need thereof, comprising administering to the subject:
verinurad or a pharmaceutically acceptable salt thereof;
a xanthine oxidase inhibitor; and
dapagliflozin.

3. The method of claim 2, wherein the condition is gout, a recurrent gout attack, gouty arthritis, hypertension, a cardiovascular disease, coronary heart disease, heart failure, Lesch-Nyhan syndrome, Kelley-Seegmiller syndrome, kidney disease, chronic kidney disease, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency or a combination thereof.

4. The method of claim 2, wherein the condition is gout.

5. The method of claim 2, wherein the condition is chronic kidney disease.

6. The method of claim 2, wherein the condition is heart failure.

7. The method of any one of claims 1-6, wherein the xanthine oxidase inhibitor is febuxostat or a pharmaceutically acceptable salt thereof.

8. The method of any one of claims 1-6, wherein the xanthine oxidase inhibitor is allopurinol or a pharmaceutically acceptable salt thereof.

9. A method of treating or preventing chronic kidney disease in a subject in need thereof, comprising administering to the subject:
verinurad or a pharmaceutically acceptable salt thereof;
allopurinol or a pharmaceutically acceptable salt thereof; and
dapagliflozin.

10. A method of treating or preventing heart failure in a subject in need thereof, comprising administering to the subject:
verinurad or a pharmaceutically acceptable salt thereof;
allopurinol or a pharmaceutically acceptable salt thereof; and
dapagliflozin.

11. A method of reducing serum uric acid levels in a subject who is currently being treated with dapagliflozin, comprising administering to the subject:
verinurad or a pharmaceutically acceptable salt thereof; and
a xanthine oxidase inhibitor.

12. The method of claim 11, wherein the subject has diabetes mellitus.

13. The method of claim 11 or 12, wherein the xanthine oxidase inhibitor is febuxostat or a pharmaceutically acceptable salt thereof.

14. The method of claim 11 or 12, wherein the xanthine oxidase inhibitor is allopurinol or a pharmaceutically acceptable salt thereof.

15. A method of treating or preventing a condition associated with hyperuricemia in a subject who is currently being treated with dapagliflozin, comprising administering to the subject:
verinurad or a pharmaceutically acceptable salt thereof; and
a xanthine oxidase inhibitor.

16. The method of claim 15, wherein the subject has diabetes mellitus.

17. The method of claim 15, wherein the condition is gout, a recurrent gout attack, gouty arthritis, hypertension, a cardiovascular disease, coronary heart disease, heart failure, Lesch-Nyhan syndrome, Kelley-Seegmiller syndrome, kidney disease, chronic kidney disease, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency or a combination thereof.

18. The method of claim 16, wherein the condition is gout, a recurrent gout attack, gouty arthritis, hypertension, a cardiovascular disease, coronary heart disease, heart failure, Lesch-Nyhan syndrome, Kelley-Seegmiller syndrome, kidney disease, chronic kidney disease, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency or a combination thereof.

19. The method of claim 15, wherein the condition is gout.

20. The method of claim 16, wherein the condition is gout.

21. The method of claim 15, wherein the condition is chronic kidney disease.

22. The method of claim 16, wherein the condition is chronic kidney disease.

23. The method of claim 15, wherein the condition is heart failure.

24. The method of claim 16, wherein the condition is heart failure.

25. The method of any one of claims 15-24, wherein the xanthine oxidase inhibitor is febuxostat or a pharmaceutically acceptable salt thereof.

26. The method of any one of claims 15-24, wherein the xanthine oxidase inhibitor is allopurinol or a pharmaceutically acceptable salt thereof.

27. A method of treating or preventing chronic kidney disease in a subject with diabetes mellitus who is currently being treated with dapagliflozin, comprising administering to the subject:
verinurad or a pharmaceutically acceptable salt thereof; and
allopurinol or a pharmaceutically acceptable salt thereof.

28. A method of treating or preventing heart failure in a subject with diabetes mellitus who is currently being treated with dapagliflozin, comprising administering to the subject:
verinurad or a pharmaceutically acceptable salt thereof; and
allopurinol or a pharmaceutically acceptable salt thereof.

29. A pharmaceutical composition comprising:
verinurad or a pharmaceutically acceptable salt thereof;
a xanthine oxidase inhibitor;
dapagliflozin; and
a pharmaceutically acceptable excipient or carrier.

30. The pharmaceutical composition of claim 29, wherein the xanthine oxidase inhibitor is febuxostat, or a pharmaceutically acceptable salt thereof.

31. The pharmaceutical composition of claim 29, wherein the xanthine oxidase inhibitor is allopurinol, or a pharmaceutically acceptable salt thereof.

32. A method of reducing serum uric acid levels in a subject in need thereof, comprising administering to the subject an effective amount of the pharmaceutical composition of any one of claims 29-31.

33. A method of treating or preventing a condition associated with hyperuricemia in a subject in need thereof, comprising administering to the subject an effective amount of the pharmaceutical composition of any one of claims 29-31.

34. The method of claim 33, wherein the condition is gout, a recurrent gout attack, gouty arthritis, hypertension, a cardiovascular disease, coronary heart disease, heart failure, Lesch-Nyhan syndrome, Kelley-Seegmiller syndrome, kidney disease, chronic kidney disease, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency or a combination thereof.

35. The method of claim 33, wherein the condition is gout.

36. The method of claim 33, wherein the condition is chronic kidney disease.

37. The method of claim 33, wherein the condition is heart failure.
